SEQUENCE LISTING (156 sequences)

SEQ ID NO: 1 - DNA, 44 bp, synthetic construct
  source 1..44: mol_type = other DNA
ctaaagaggagaaaggatctnnkgacaaaaagtactctattggc

SEQ ID NO: 2 - DNA, 41 bp, synthetic construct
  source 1..41: mol_type = other DNA
agaggagaaaggatctatgnnkaaaaagtactctattggcc

SEQ ID NO: 3 - DNA, 38 bp, synthetic construct
  source 1..38: mol_type = other DNA
agaaaggatctatggacnnkaagtactctattggcctg

SEQ ID NO: 4 - DNA, 37 bp, synthetic construct
  source 1..37: mol_type = other DNA
aaggatctatggacaaannktactctattggcctgga

SEQ ID NO: 5 - DNA, 39 bp, synthetic construct
  source 1..39: mol_type = other DNA
ggatctatggacaaaaagnnktctattggcctggatatc

SEQ ID NO: 6 - DNA, 37 bp, synthetic construct
  source 1..37: mol_type = other DNA
ctatggacaaaaagtacnnkattggcctggatatcgg

SEQ ID NO: 7 - DNA, 33 bp, synthetic construct
  source 1..33: mol_type = other DNA
gacaaaaagtactctnnkggcctggatatcggg

SEQ ID NO: 8 - DNA, 39 bp, synthetic construct
  source 1..39: mol_type = other DNA
gacaaaaagtactctattnnkctggatatcgggaccaac

SEQ ID NO: 9 - DNA, 35 bp, synthetic construct
  source 1..35: mol_type = other DNA
aaagtactctattggcnnkgatatcgggaccaaca

SEQ ID NO: 10 - DNA, 30 bp, synthetic construct
  source 1..30: mol_type = other DNA
ctctattggcctgnnkatcgggaccaacag

SEQ ID NO: 11 - DNA, 25 bp, synthetic construct
  source 1..25: mol_type = other DNA
ttggcctggatnnkgggaccaacag

SEQ ID NO: 12 - DNA, 27 bp, synthetic construct
  source 1..27: mol_type = other DNA
ggcctggatatcnnkaccaacagcgtc

SEQ ID NO: 13 - DNA, 27 bp, synthetic construct
  source 1..27: mol_type = other DNA
ctggatatcgggnnkaacagcgtcggg

SEQ ID NO: 14 - DNA, 25 bp, synthetic construct
  source 1..25: mol_type = other DNA
atatcgggaccnnkagcgtcgggtg

SEQ ID NO: 15 - DNA, 25 bp, synthetic construct
  source 1..25: mol_type = other DNA
tcgggaccaacnnkgtcgggtgggc

SEQ ID NO: 16 - DNA, 25 bp, synthetic construct
  source 1..25: mol_type = other DNA
ggaccaacagcnnkgggtgggctgt

SEQ ID NO: 17 - DNA, 29 bp, synthetic construct
  source 1..29: mol_type = other DNA
gaccaacagcgtcnnktgggctgttatca

SEQ ID NO: 18 - DNA, 25 bp, synthetic construct
  source 1..25: mol_type = other DNA
acagcgtcgggnnkgctgttatcac

SEQ ID NO: 19 - DNA, 25 bp, synthetic construct
  source 1..25: mol_type = other DNA
gcgtcgggtggnnkgttatcaccga

SEQ ID NO: 20 - DNA, 25 bp, synthetic construct
  source 1..25: mol_type = other DNA
tcgggtgggctnnkatcaccgacga

SEQ ID NO: 21 - DNA, 28 bp, synthetic construct
  source 1..28: mol_type = other DNA
gggtgggctgttnnkaccgacgagtata

SEQ ID NO: 22 - DNA, 34 bp, synthetic construct
  source 1..34: mol_type = other DNA
gggtgggctgttatcnnkgacgagtataaagtac

SEQ ID NO: 23 - DNA, 36 bp, synthetic construct
  source 1..36: mol_type = other DNA
gtgggctgttatcaccnnkgagtataaagtaccttc

SEQ ID NO: 24 - DNA, 36 bp, synthetic construct
  source 1..36: mol_type = other DNA
ggctgttatcaccgacnnktataaagtaccttcgaa

SEQ ID NO: 25 - DNA, 37 bp, synthetic construct
  source 1..37: mol_type = other DNA
ctgttatcaccgacgagnnkaaagtaccttcgaaaaa

SEQ ID NO: 26 - DNA, 38 bp, synthetic construct
  source 1..38: mol_type = other DNA
ttatcaccgacgagtatnnkgtaccttcgaaaaagttc

SEQ ID NO: 27 - DNA, 37 bp, synthetic construct
  source 1..37: mol_type = other DNA
tcaccgacgagtataaannkccttcgaaaaagttcaa

SEQ ID NO: 28 - DNA, 40 bp, synthetic construct
  source 1..40: mol_type = other DNA
accgacgagtataaagtannktcgaaaaagttcaaagtgc

SEQ ID NO: 29 - DNA, 41 bp, synthetic construct
  source 1..41: mol_type = other DNA
cgacgagtataaagtacctnnkaaaaagttcaaagtgctgg

SEQ ID NO: 30 - DNA, 37 bp, synthetic construct
  source 1..37: mol_type = other DNA
agtataaagtaccttcgnnkaagttcaaagtgctggg

SEQ ID NO: 31 - DNA, 37 bp, synthetic construct
  source 1..37: mol_type = other DNA
ataaagtaccttcgaaannkttcaaagtgctgggcaa

SEQ ID NO: 32 - DNA, 33 bp, synthetic construct
  source 1..33: mol_type = other DNA
gtaccttcgaaaaagnnkaaagtgctgggcaac

SEQ ID NO: 33 - DNA, 30 bp, synthetic construct
  source 1..30: mol_type = other DNA
ttcgaaaaagttcnnkgtgctgggcaacac

SEQ ID NO: 34 - DNA, 32 bp, synthetic construct
  source 1..32: mol_type = other DNA
cgaaaaagttcaaannkctgggcaacaccgat

SEQ ID NO: 35 - DNA, 31 bp, synthetic construct
  source 1..31: mol_type = other DNA
aaaagttcaaagtgnnkggcaacaccgatcg

SEQ ID NO: 36 - DNA, 29 bp, synthetic construct
  source 1..29: mol_type = other DNA
gttcaaagtgctgnnkaacaccgatcgcc

SEQ ID NO: 37 - DNA, 25 bp, synthetic construct
  source 1..25: mol_type = other DNA
aagtgctgggcnnkaccgatcgcca

SEQ ID NO: 38 - DNA, 25 bp, synthetic construct
  source 1..25: mol_type = other DNA
tgctgggcaacnnkgatcgccattc

SEQ ID NO: 39 - DNA, 27 bp, synthetic construct
  source 1..27: mol_type = other DNA
ctgggcaacaccnnkcgccattcaatc

SEQ ID NO: 40 - DNA, 34 bp, synthetic construct
  source 1..34: mol_type = other DNA
ctgggcaacaccgatnnkcattcaatcaaaaaga

SEQ ID NO: 41 - DNA, 32 bp, synthetic construct
  source 1..32: mol_type = other DNA
gcaacaccgatcgcnnktcaatcaaaaagaac

SEQ ID NO: 42 - DNA, 36 bp, synthetic construct
  source 1..36: mol_type = other DNA
caacaccgatcgccatnnkatcaaaaagaacttgat

SEQ ID NO: 43 - DNA, 35 bp, synthetic construct
  source 1..35: mol_type = other DNA
caccgatcgccattcannkaaaaagaacttgattg

SEQ ID NO: 44 - DNA, 35 bp, synthetic construct
  source 1..35: mol_type = other DNA
cgatcgccattcaatcnnkaagaacttgattggtg

SEQ ID NO: 45 - DNA, 33 bp, synthetic construct
  source 1..33: mol_type = other DNA
cgccattcaatcaaannkaacttgattggtgcg

SEQ ID NO: 46 - DNA, 34 bp, synthetic construct
  source 1..34: mol_type = other DNA
cattcaatcaaaaagnnkttgattggtgcgctgt

SEQ ID NO: 47 - DNA, 37 bp, synthetic construct
  source 1..37: mol_type = other DNA
attcaatcaaaaagaacnnkattggtgcgctgttgtt

SEQ ID NO: 48 - DNA, 33 bp, synthetic construct
  source 1..33: mol_type = other DNA
atcaaaaagaacttgnnkggtgcgctgttgttt

SEQ ID NO: 49 - DNA, 37 bp, synthetic construct
  source 1..37: mol_type = other DNA
tcaaaaagaacttgattnnkgcgctgttgtttgactc

SEQ ID NO: 50 - DNA, 35 bp, synthetic construct
  source 1..35: mol_type = other DNA
aaagaacttgattggtnnkctgttgtttgactccg

SEQ ID NO: 51 - DNA, 30 bp, synthetic construct
  source 1..30: mol_type = other DNA
cttgattggtgcgnnkttgtttgactccgg

SEQ ID NO: 52 - DNA, 25 bp, synthetic construct
  source 1..25: mol_type = other DNA
ttggtgcgctgnnktttgactccgg

SEQ ID NO: 53 - DNA, 25 bp, synthetic construct
  source 1..25: mol_type = other DNA
gtgcgctgttgnnkgactccgggga

SEQ ID NO: 54 - DNA, 26 bp, synthetic construct
  source 1..26: mol_type = other DNA
cgctgttgtttnnktccggggaaacc

SEQ ID NO: 55 - DNA, 27 bp, synthetic construct
  source 1..27: mol_type = other DNA
ctgttgtttgacnnkggggaaaccgcc

SEQ ID NO: 56 - DNA, 27 bp, synthetic construct
  source 1..27: mol_type = other DNA
ttgtttgactccnnkgaaaccgccgag

SEQ ID NO: 57 - DNA, 25 bp, synthetic construct
  source 1..25: mol_type = other DNA
ttgactccgggnnkaccgccgaggc

SEQ ID NO: 58 - DNA, 25 bp, synthetic construct
  source 1..25: mol_type = other DNA
actccggggaannkgccgaggcgac

SEQ ID NO: 59 - DNA, 25 bp, synthetic construct
  source 1..25: mol_type = other DNA
ccggggaaaccnnkgaggcgactcg

SEQ ID NO: 60 - DNA, 25 bp, synthetic construct
  source 1..25: mol_type = other DNA
gggaaaccgccnnkgcgactcgcct

SEQ ID NO: 61 - DNA, 28 bp, synthetic construct
  source 1..28: mol_type = other DNA
gaaaccgccgagnnkactcgccttaaac

SEQ ID NO: 62 - DNA, 25 bp, synthetic construct
  source 1..25: mol_type = other DNA
ccgccgaggcgnnkcgccttaaacg

SEQ ID NO: 63 - DNA, 28 bp, synthetic construct
  source 1..28: mol_type = other DNA
gccgaggcgactnnkcttaaacgtacag

SEQ ID NO: 64 - DNA, 25 bp, synthetic construct
  source 1..25: mol_type = other DNA
aggcgactcgcnnkaaacgtacagc

SEQ ID NO: 65 - DNA, 25 bp, synthetic construct
  source 1..25: mol_type = other DNA
cgactcgccttnnkcgtacagcacg

SEQ ID NO: 66 - DNA, 27 bp, synthetic construct
  source 1..27: mol_type = other DNA
actcgccttaaannkacagcacgtcgc

SEQ ID NO: 67 - DNA, 25 bp, synthetic construct
  source 1..25: mol_type = other DNA
gccttaaacgtnnkgcacgtcgccg

SEQ ID NO: 68 - DNA, 29 bp, synthetic construct
  source 1..29: mol_type = other DNA
ccttaaacgtacannkcgtcgccggtaca

SEQ ID NO: 69 - DNA, 29 bp, synthetic construct
  source 1..29: mol_type = other DNA
taaacgtacagcannkcgccggtacactc

SEQ ID NO: 70 - DNA, 26 bp, synthetic construct
  source 1..26: mol_type = other DNA
gtacagcacgtnnkcggtacactcgg

SEQ ID NO: 71 - DNA, 25 bp, synthetic construct
  source 1..25: mol_type = other DNA
cagcacgtcgcnnktacactcggcg

SEQ ID NO: 72 - DNA, 25 bp, synthetic construct
  source 1..25: mol_type = other DNA
cacgtcgccggnnkactcggcgtaa

SEQ ID NO: 73 - DNA, 25 bp, synthetic construct
  source 1..25: mol_type = other DNA
gtcgccggtacnnkcggcgtaagaa

SEQ ID NO: 74 - DNA, 27 bp, synthetic construct
  source 1..27: mol_type = other DNA
cgccggtacactnnkcgtaagaatcgc

SEQ ID NO: 75 - DNA, 30 bp, synthetic construct
  source 1..30: mol_type = other DNA
ccggtacactcggnnkaagaatcgcatttg

SEQ ID NO: 76 - DNA, 30 bp, synthetic construct
  source 1..30: mol_type = other DNA
gtacactcggcgtnnkaatcgcatttgcta

SEQ ID NO: 77 - DNA, 30 bp, synthetic construct
  source 1..30: mol_type = other DNA
cactcggcgtaagnnkcgcatttgctattt

SEQ ID NO: 78 - DNA, 35 bp, synthetic construct
  source 1..35: mol_type = other DNA
cactcggcgtaagaatnnkatttgctatttgcagg

SEQ ID NO: 79 - DNA, 30 bp, synthetic construct
  source 1..30: mol_type = other DNA
gcgtaagaatcgcnnktgctatttgcagga

SEQ ID NO: 80 - DNA, 37 bp, synthetic construct
  source 1..37: mol_type = other DNA
ggcgtaagaatcgcattnnktatttgcaggaaatctt

SEQ ID NO: 81 - DNA, 38 bp, synthetic construct
  source 1..38: mol_type = other DNA
gtaagaatcgcatttgcnnkttgcaggaaatctttagc

SEQ ID NO: 82 - DNA, 38 bp, synthetic construct
  source 1..38: mol_type = other DNA
agaatcgcatttgctatnnkcaggaaatctttagcaac

SEQ ID NO: 83 - DNA, 37 bp, synthetic construct
  source 1..37: mol_type = other DNA
atcgcatttgctatttgnnkgaaatctttagcaacga

SEQ ID NO: 84 - DNA, 37 bp, synthetic construct
  source 1..37: mol_type = other DNA
gcatttgctatttgcagnnkatctttagcaacgagat

SEQ ID NO: 85 - DNA, 35 bp, synthetic construct
  source 1..35: mol_type = other DNA
ttgctatttgcaggaannktttagcaacgagatgg

SEQ ID NO: 86 - DNA, 31 bp, synthetic construct
  source 1..31: mol_type = other DNA
atttgcaggaaatcnnkagcaacgagatggc

SEQ ID NO: 87 - DNA, 37 bp, synthetic construct
  source 1..37: mol_type = other DNA
atttgcaggaaatctttnnkaacgagatggcaaaagt

SEQ ID NO: 88 - DNA, 44 bp, synthetic construct
  source 1..44: mol_type = other DNA
gtggggatagcggtggtagcgatggtggttcaagcggtagcgaa

SEQ ID NO: 89 - DNA, 44 bp, synthetic construct
  source 1..44: mol_type = other DNA
ttcgctaccgcttgaaccaccatcgctaccaccgctatccccac

SEQ ID NO: 90 - DNA, 44 bp, synthetic construct
  source 1..44: mol_type = other DNA
gtggggatagcggtggtagcaaaggtggttcaagcggtagcgaa

SEQ ID NO: 91 - DNA, 44 bp, synthetic construct
  source 1..44: mol_type = other DNA
ttcgctaccgcttgaaccacctttgctaccaccgctatccccac

SEQ ID NO: 92 - DNA, 46 bp, synthetic construct
  source 1..46: mol_type = other DNA
gtggtagcagcggtggttcagcgggtagcgaaacaccgggtacaag

SEQ ID NO: 93 - DNA, 46 bp, synthetic construct
  source 1..46: mol_type = other DNA
cttgtacccggtgtttcgctacccgctgaaccaccgctgctaccac

SEQ ID NO: 94 - DNA, 45 bp, synthetic construct
  source 1..45: mol_type = other DNA
agcggtggttcaagcggtattgaaacaccgggtacaagcgaaagc

SEQ ID NO: 95 - DNA, 45 bp, synthetic construct
  source 1..45: mol_type = other DNA
gctttcgcttgtacccggtgtttcaataccgcttgaaccaccgct

SEQ ID NO: 96 - DNA, 48 bp, synthetic construct
  source 1..48: mol_type = other DNA
gtggttcaagcggtagcgaaacagatggtacaagcgaaagcgcaacac

SEQ ID NO: 97 - DNA, 48 bp, synthetic construct
  source 1..48: mol_type = other DNA
gtgttgcgctttcgcttgtaccatctgtttcgctaccgcttgaaccac

SEQ ID NO: 98 - DNA, 42 bp, synthetic construct
  source 1..42: mol_type = other DNA
agcgaaacaccgggtacaagcaaaagcgcaacaccggaaagc

SEQ ID NO: 99 - DNA, 42 bp, synthetic construct
  source 1..42: mol_type = other DNA
gctttccggtgttgcgcttttgcttgtacccggtgtttcgct

SEQ ID NO: 100 - DNA, 45 bp, synthetic construct
  source 1..45: mol_type = other DNA
agcgcaacaccggaaagcgcgggtggtagctcaggtggtagtagc

SEQ ID NO: 101 - DNA, 45 bp, synthetic construct
  source 1..45: mol_type = other DNA
gctactaccacctgagctaccacccgcgctttccggtgttgcgct

SEQ ID NO: 102 - DNA, 58 bp, synthetic construct
  source 1..58: mol_type = other DNA
ccggaaagcagtggtggtagccgtggtggtagtagcactttaaatattgaggatgagc

SEQ ID NO: 103 - DNA, 58 bp, synthetic construct
  source 1..58: mol_type = other DNA
gctcatcctcaatatttaaagtgctactaccaccacggctaccaccactgctttccgg

SEQ ID NO: 104 - DNA, 56 bp, synthetic construct
  source 1..56: mol_type = other DNA
agcagtggtggtagctcaggtaccagtagcactttaaatattgaggatgagcatcg

SEQ ID NO: 105 - DNA, 56 bp, synthetic construct
  source 1..56: mol_type = other DNA
cgatgctcatcctcaatatttaaagtgctactggtacctgagctaccaccactgct

SEQ ID NO: 106 - DNA, 60 bp, synthetic construct
  source 1..60: mol_type = other DNA
ctcaggtggtagtagcactttaaatattcgtgatgagcatcgtttacatgagacatcaaa

SEQ ID NO: 107 - DNA, 60 bp, synthetic construct
  source 1..60: mol_type = other DNA
tttgatgtctcatgtaaacgatgctcatcacgaatatttaaagtgctactaccacctgag

SEQ ID NO: 108 - DNA, 60 bp, synthetic construct
  source 1..60: mol_type = other DNA
ctcaggtggtagtagcactttaaatattagcgatgagcatcgtttacatgagacatcaaa

SEQ ID NO: 109 - DNA, 60 bp, synthetic construct
  source 1..60: mol_type = other DNA
tttgatgtctcatgtaaacgatgctcatcgctaatatttaaagtgctactaccacctgag

SEQ ID NO: 110 - DNA, 60 bp, synthetic construct
  source 1..60: mol_type = other DNA
tcaggtggtagtagcactttaaatattgagagcgagcatcgtttacatgagacatcaaaa

SEQ ID NO: 111 - DNA, 60 bp, synthetic construct
  source 1..60: mol_type = other DNA
ttttgatgtctcatgtaaacgatgctcgctctcaatatttaaagtgctactaccacctga

SEQ ID NO: 112 - DNA, 60 bp, synthetic construct
  source 1..60: mol_type = other DNA
gtggtagtagcactttaaatattgaggattttcatcgtttacatgagacatcaaaagaac

SEQ ID NO: 113 - DNA, 60 bp, synthetic construct
  source 1..60: mol_type = other DNA
gttcttttgatgtctcatgtaaacgatgaaaatcctcaatatttaaagtgctactaccac

SEQ ID NO: 114 - DNA, 60 bp, synthetic construct
  source 1..60: mol_type = other DNA
gtggtagtagcactttaaatattgaggatctgcatcgtttacatgagacatcaaaagaac

SEQ ID NO: 115 - DNA, 60 bp, synthetic construct
  source 1..60: mol_type = other DNA
gttcttttgatgtctcatgtaaacgatgcagatcctcaatatttaaagtgctactaccac

SEQ ID NO: 116 - DNA, 60 bp, synthetic construct
  source 1..60: mol_type = other DNA
agtagcactttaaatattgaggatgagcatattttacatgagacatcaaaagaacccgac

SEQ ID NO: 117 - DNA, 60 bp, synthetic construct
  source 1..60: mol_type = other DNA
gtcgggttcttttgatgtctcatgtaaaatatgctcatcctcaatatttaaagtgctact

SEQ ID NO: 118 - DNA, 52 bp, synthetic construct
  source 1..52: mol_type = other DNA
ttgaggatgagcatcgtttacatgagtattcaaaagaacccgacgtgagctt

SEQ ID NO: 119 - DNA, 52 bp, synthetic construct
  source 1..52: mol_type = other DNA
aagctcacgtcgggttcttttgaatactcatgtaaacgatgctcatcctcaa

SEQ ID NO: 120 - DNA, 54 bp, synthetic construct
  source 1..54: mol_type = other DNA
ggatgagcatcgtttacatgagacatcaaaaccgcccgacgtgagcttagggtc

SEQ ID NO: 121 - DNA, 54 bp, synthetic construct
  source 1..54: mol_type = other DNA
gaccctaagctcacgtcgggcggttttgatgtctcatgtaaacgatgctcatcc

SEQ ID NO: 122 - DNA, 56 bp, synthetic construct
  source 1..56: mol_type = other DNA
tcgtttacatgagacatcaaaagaacccgactttagcttagggtcaacgtggcttt

SEQ ID NO: 123 - DNA, 56 bp, synthetic construct
  source 1..56: mol_type = other DNA
aaagccacgttgaccctaagctaaagtcgggttcttttgatgtctcatgtaaacga

SEQ ID NO: 124 - DNA, 56 bp, synthetic construct
  source 1..56: mol_type = other DNA
tcgtttacatgagacatcaaaagaacccgaccatagcttagggtcaacgtggcttt

SEQ ID NO: 125 - DNA, 56 bp, synthetic construct
  source 1..56: mol_type = other DNA
aaagccacgttgaccctaagctatggtcgggttcttttgatgtctcatgtaaacga

SEQ ID NO: 126 - DNA, 52 bp, synthetic construct
  source 1..52: mol_type = other DNA
tgagacatcaaaagaacccgacgtgtatttagggtcaacgtggctttctgac

SEQ ID NO: 127 - DNA, 52 bp, synthetic construct
  source 1..52: mol_type = other DNA
gtcagaaagccacgttgaccctaaatacacgtcgggttcttttgatgtctca

SEQ ID NO: 128 - DNA, 54 bp, synthetic construct
  source 1..54: mol_type = other DNA
acatcaaaagaacccgacgtgagcttacattcaacgtggctttctgacttcccc

SEQ ID NO: 129 - DNA, 54 bp, synthetic construct
  source 1..54: mol_type = other DNA
ggggaagtcagaaagccacgttgaatgtaagctcacgtcgggttcttttgatgt

SEQ ID NO: 130 - DNA, 40 bp, synthetic construct
  source 1..40: mol_type = other DNA
ggcgtgggcggagactcgtggaatggggttagctgtccgc

SEQ ID NO: 131 - DNA, 40 bp, synthetic construct
  source 1..40: mol_type = other DNA
gcggacagctaaccccattccacgagtctccgcccacgcc

SEQ ID NO: 132 - DNA, 50 bp, synthetic construct
  source 1..50: mol_type = other DNA
gggttagctgtccgccaagcaaccttgatcatcccgttaaaggcaacgtc

SEQ ID NO: 133 - DNA, 50 bp, synthetic construct
  source 1..50: mol_type = other DNA
gacgttgcctttaacgggatgatcaaggttgcttggcggacagctaaccc

SEQ ID NO: 134 - DNA, 49 bp, synthetic construct
  source 1..49: mol_type = other DNA
gcggcagcagcaccctaaatataagagatgagcaccggctacatgagac

SEQ ID NO: 135 - DNA, 49 bp, synthetic construct
  source 1..49: mol_type = other DNA
gtctcatgtagccggtgctcatctcttatatttagggtgctgctgccgc

SEQ ID NO: 136 - DNA, 54 bp, synthetic construct
  source 1..54: mol_type = other DNA
gctacatgagacctcaaaagagccagatttctctctagggtccacatggctgtc

SEQ ID NO: 137 - DNA, 54 bp, synthetic construct
  source 1..54: mol_type = other DNA
gacagccatgtggaccctagagagaaatctggctcttttgaggtctcatgtagc

SEQ ID NO: 138 - DNA, 44 bp, synthetic construct
  source 1..44: mol_type = other DNA
ctggaggatctagcggaggatccgccggcagcgagacaccagga

SEQ ID NO: 139 - DNA, 44 bp, synthetic construct
  source 1..44: mol_type = other DNA
tcctggtgtctcgctgccggcggatcctccgctagatcctccag

SEQ ID NO: 140 - DNA, 34 bp, synthetic construct
  source 1..34: mol_type = other DNA
gagcagtggcggcagcctgggcggcagcagcacc

SEQ ID NO: 141 - DNA, 34 bp, synthetic construct
  source 1..34: mol_type = other DNA
ggtgctgctgccgcccaggctgccgccactgctc

SEQ ID NO: 142 - DNA, 50 bp, synthetic construct
  source 1..50: mol_type = other DNA
cggcagcagcaccctaaatatagaaagcgagcaccggctacatgagacct

SEQ ID NO: 143 - DNA, 50 bp, synthetic construct
  source 1..50: mol_type = other DNA
aggtctcatgtagccggtgctcgctttctatatttagggtgctgctgccg

SEQ ID NO: 144 - DNA, 61 bp, synthetic construct
  source 1..61: mol_type = other DNA
tgagacctcaaaagagccagatgtttctctacactccacatggctgtctgattttcctcag

SEQ ID NO: 145 - DNA, 61 bp, synthetic construct
  source 1..61: mol_type = other DNA
ctgaggaaaatcagacagccatgtggagtgtagagaaacatctggctcttttgaggtctca

SEQ ID NO: 146 - DNA, 39 bp, synthetic construct
  source 1..39: mol_type = other DNA
cgagtcagcaacaccagagagcgccggcggcagcagcgg

SEQ ID NO: 147 - DNA, 39 bp, synthetic construct
  source 1..39: mol_type = other DNA
ccgctgctgccgccggcgctctctggtgttgctgactcg

SEQ ID NO: 148 - DNA, 57 bp, synthetic construct
  source 1..57: mol_type = other DNA
cggcagcagcaccctaaatatagaagatttccaccggctacatgagacctcaaaaga

SEQ ID NO: 149 - DNA, 57 bp, synthetic construct
  source 1..57: mol_type = other DNA
tcttttgaggtctcatgtagccggtggaaatcttctatatttagggtgctgctgccg

SEQ ID NO: 150 - DNA, 22 bp, synthetic construct
  source 1..22: mol_type = other DNA
agggacgactttagaccttaga

SEQ ID NO: 151 - DNA, 21 bp, synthetic construct
  source 1..21: mol_type = other DNA
gtctctgaccactgcgatatg

SEQ ID NO: 152 - protein, 1368 aa, synthetic construct
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 153 - protein, 677 aa, synthetic construct
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGLLTSEGKEIKNKDEILALLKALFLPKRLSIIHCPGHQKGHSAEARGNRMADQAARKAAITETPDTSTLLIENSSP

SEQ ID NO: 154 - protein, 677 aa, synthetic construct
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTGGSSLLQEGQRKAGAAVTTETEVIWAKALPAGTSAQRAQLIALTQALKMAEGKKLNVYTNSRYAFATAHIHGEIYRRRGLLTSEGKEIKNKDEILALLKALFLPKRLSIIHCPGHQKGHSAEARGNRMADQAARKAAITETPDTSTLLIENSSP

SEQ ID NO: 155 - protein, 677 aa, synthetic construct
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSP

SEQ ID NO: 156 - protein, 677 aa, synthetic construct
TLNIEDEYRLHETSKEPDVSLGSTWLSDFPQAWAETGGMGLAVRQAPLIIPLKATSTPVSIKQYPMSREARLGIKPHIRRLYDQGILVPCQSPWNTPLRPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWITDARKETVMGQPTPKTPRELREFLGKAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLNILAPHAVEALVKQPPDRWLSNARMTHYQALLLDTDRVQFGPVVALNPATLLPLPEEGLQHNCLDILAEAHGTRPDLTDQPLPDADHTWYTGGSSLLQEGQRKAGAAVTTETEVIWAKALPAGTSAQRAQLIALTQALKMAEGKKLNVYTNSRYAFATAHWHGEIYRRRGLLTSEGKEIKNKDEILALLKALFLPKRLSIIHCPGHQKGHSAEARGNRMADQAARKAAITETPDTSTLLIENSSP

(19) United States
(12) Patent Application Publication
(10) Pub. No.: US 2026/0193630 A1
(43) Pub. Date: Jul. 9, 2026

(54) CAS9 and Reverse Transcriptase Mutants with Improved Activity in Prime Editing Applications

(71) Applicant: Integrated DNA Technologies, Inc., Coralville, IA (US)

(72) Inventors: Christopher Anthony VAKULSKAS, North Liberty, IA (US); Sarah Franz BEAUDOIN, Iowa City, IA (US); Michael Allen COLLINGWOOD, Solon, IA (US); Diane DEZWAAN, Swisher, IA (US); Adam BRAINARD, Cedar Rapids, IA (US); Katherine KEOGH, Coralville, IA (US); Susan Marie RUPP, Marion, IA (US)

(21) Appl. No.: 19/262,179

(22) Filed: Jul. 8, 2025

Related U.S. Application Data
(60) Provisional application No. 63/668,607, filed on Jul. 8, 2024.

(51) Int. Cl. C12N 9/22; C12N 9/12; C12N 15/113; C12N 15/52; C12N 15/62; C12N 15/74; C12N 15/79

(52) U.S. Cl. CPC C12N 9/226; C12N 9/1276; C12N 15/113; C12N 15/52; C12N 15/62; C12N 15/74; C12N 15/79; C12Y 207/07049; C12N 2310/20; C12N 2800/101

(57) ABSTRACT

This invention pertains to fusion protein mutants comprising a Prime Editing enzyme having a first amino acid sequence and a second amino acid sequence, wherein the first amino acid sequence comprises a SpCas9 H840A nickase mutant protein of SEQ ID NO:152 and the second amino acid sequence comprises a Moloney Murine Leukemia Virus reverse transcriptase protein mutant (MMLV RTase mutant), wherein the fusion protein mutant displays at least the equivalent or greater activity of a reference Prime Editing enzyme in genome editing.

This application claim benefit of U.S. Ser. No. 63/668,607 filed Jul. 8, 2024, the entirety of which is incorporated by reference in its entirety.

### SEQUENCE LISTING

The instant application contains a Sequence Listing that has been submitted in XML format via Patent Center and is hereby incorporated by reference in its entirety. The XML copy, created on Jul. 8, 2025, is named 6391-0023US01_Sequence.xml, and is 145,265 bytes in size.

### FIELD OF THE INVENTION

This invention pertains to the ability of a nickase CRISPR/Cas9 mutant to cleave double-stranded DNA on one strand in a targeted manner in living cells when complexed with sgRNAs.

### BACKGROUND OF THE INVENTION

SpCas9 is an RNA guided endonuclease from the Clustered Regularly Interspaced Short Palindromic Repeat (CRISPR)-Cas (CRISPR-associated) bacterial adaptive immune system of Streptococcus pyogenes [1]. Cas9 is guided to a 23-nt DNA target sequence by a target site-specific 20-nt complementary RNA (part of the 44-nt crRNA) and a universal 89-nt tracrRNA, collectively referred to as the guide RNA (gRNA) complex. The Cas9-gRNA ribonucleoprotein (RNP) complex mediates double-stranded DNA breaks (DSBs) which are then typically repaired by the non-homologous end joining (NHEJ), microhomology mediated end joining, or homology-directed repair (HDR) system if a suitable template nucleic acid is present.

S. pyogenes Cas9 protein contains two endonuclease domains that function together to generate a double-strand DNA break by cleaving both the target (guide complementary) and non-target (guide noncomplementary) strands of a double-stranded DNA (dsDNA). These conserved domains are the RuvC and HNH domains. There are two known mutations that can alter Cas9, which produces a double-stranded cut, into a ‘Nickase’ that results in single-stranded cuts. Cas9 D10A variant generates the nick on the targeted strand, while the Cas9 H840A variant generates the nick on the non-targeted strand [1]. The nickase Cas9 variants have been used to facilitate CRISPR-targeted genome editing approaches that do not rely on the introduction of a dsDNA break, examples of which include cytosine/adenine base editors [2,3] and more recently the Cas9 prime editor [4].

Prime Editing is a new technology that utilizes a Cas9 nickase fused to an engineered reverse transcriptase. The fusion is coupled with a prime editing guide RNA (pegRNA) that recognizes the target site and contains the desired edit. The prime editor was developed by David Liu and it contains the Cas9 nickase, H840A, and a highly mutagenized reverse transcriptase from Moloney Murine Leukemia Virus (MMLV RTase). The engineered RTase is derived from multiple patents that would require expensive licenses for use and sale of this technology [4].

There is a long-felt need to improve prime editor capabilities through discovery of novel mutations within the Cas9 nickase and MMLV RTase.

### BRIEF SUMMARY OF THE INVENTION

This invention pertains to the ability to create a genomic/DNA change utilizing a a SpCas9 H840A nickase mutant and a Reverse Transcriptase (RT) in a method known as “Prime Editing.”

In a first aspect, a fusion protein mutant including a Prime Editing enzyme is provided. The Prime Editing enzyme includes a first amino acid sequence and a second amino acid sequence, wherein the first amino acid sequence comprises a SpCas9 H840A nickase mutant protein of SEQ ID NO:152 and the second amino acid sequence comprises a Moloney Murine Leukemia Virus reverse transcriptase protein mutant (MMLV RTase mutant). The fusion protein mutant displays at least the equivalent or greater activity of a reference Prime Editing enzyme in genome editing.

In a second aspect, a nucleic acid sequence encoding the fusion protein of claims of the first aspect is provided.

In a third aspect, an isolated ribonucleoprotein complex is provided. The isolated ribonucleoprotein complex includes the fusion protein mutant of the first aspect and a gRNA. In a first respect, the gRNA includes a pegRNA.

In a fourth aspect, a CRISPR/Cas endonuclease system including the fusion protein mutant of the first aspect is provided. In a first respect, the CRISPR/Cas endonuclease system is encoded by a DNA expression vector.

In a fifth aspect, a method of performing gene editing in a eukaryotic cell is provided. The method includes a step of contacting a candidate editing target site locus with an active CRISPR/Cas endonuclease system having the fusion protein mutant of the first aspect

In a sixth aspect, a kit for performing gene editing in a eukaryotic cell is provided. The kit includes the fusion protein mutant of the first aspect and optionally a gRNA.

### DETAILED DESCRIPTION OF THE INVENTION

The present invention pertains to using methods to select bacterial prime editing variants having novel mutations within the Cas9 nickase and MMLV RTase possessing more potent prime editors. To our knowledge, no comprehensive screen with selection has been performed for MMLV RTase, nor the prime editor in its entirety, and the current amino acid substitutions found in the published prime editor were isolated through rational mutagenesis or random mutagenesis by error prone PCR. Focusing on the complete prime editor construct, we made 25 mutant libraries of every possible amino acid substitution spanning the entire open reading frame of the prime editor (Cas9 and MMLV RTase genes and the amino acid linker between the two proteins). We screened the libraries for substitutions that would enable prime editing in bacteria with the highest overall potency.

The term “mutant MMLV-II RTase protein” (or “Mutant MMLV-II protein”) refers to a MMLV RTase protein having three amino acid substitutions within the MMLV RTase amino acid sequence relative to the WT MMLV RTase (D524G, E562Q, and D583N; see SEQ ID NO: 154).

The term “mutant PE2 RTase protein” (or “Mutant PE2 M-MLV RT protein”) refers to a MMLV RTase protein having five amino acid substitutions within the MMLV RTase amino acid sequence (D200N; L603W; T330P; T306K; and W313F; see SEQ ID NO: 156).

The term “mutant PE2 fusion protein” refers to a Cas9 (H840A)-MMLV RTase fusion protein having five amino acid substitutions within the MMLV RTase amino acid sequence (D200N; L603W; T330P; T306K; and W313F; see SEQ ID NO: 155).

The terms, “Mutant ID,” and “ID,” as used in the disclosure refer to the change in amino acid and codon at a given position relative to a reference protein (e.g., wild-type Cas9 protein). For example, a Mutant ID or ID characterized as “D2P_GAC_CCG” refers to a mutant amino acid at position 2, where Aspartic acid in the reference protein is changed to Proline in the mutant protein, and where the corresponding codon GAC in the open reading frame of the reference protein is changed to the codon CCG in the open reading frame of the mutant protein.

The term “Cas9 (H840A) protein” encompasses a protein having the identical amino acid sequence of the naturally-occurring Streptococcus pyogenes Cas9 bearing the single amino acid substation at position 840 where an Alanine is substituted for Histidine (e.g., SEQ ID NO:152) and that has biochemical and biological activity when combined with a suitable guide RNA (for example sgRNA, dual crRNA: tracrRNA, or pegRNA compositions) to form an active CRISPR-Cas endonuclease system.

The term “CRISPR/Cas endonuclease system” refers to a CRISPR/Cas endonuclease system that includes a functional Cas9 protein or mutant thereof and a suitable gRNA.

The term “isolated nucleic acid” include DNA, RNA, cDNA, and vectors encoding the same, where the DNA, RNA, cDNA and vectors are free of other biological materials from which they may be derived or associated, such as cellular components. Typically, an isolated nucleic acid will be purified from other biological materials from which they may be derived or associated, such as cellular components.

A competent CRISPR-Cas endonuclease system includes a ribonucleoprotein (RNP) complex formed with a mutant Cas9 protein or a mutant Cas9-MMLV RTase fusion protein and an isolated guide RNA selected from one of a pegRNA, a dual crRNA: tracrRNA combination or a chimeric single-molecule sgRNA.

### Applications

In a first aspect, a fusion protein mutant including a Prime Editing enzyme is provided. The Prime Editing enzyme includes a first amino acid sequence and a second amino acid sequence, wherein the first amino acid sequence comprises a SpCas9 H840A nickase mutant protein of SEQ ID NO:152 and the second amino acid sequence comprises a Moloney Murine Leukemia Virus reverse transcriptase protein mutant (MMLV RTase mutant). The fusion protein mutant displays at least the equivalent or greater activity of a reference Prime Editing enzyme in genome editing. In a first respect, the fusion protein mutant includes a member of the group selected from one of Tables 2, 6, 7, 8, 9, 10, and 11.

In a second aspect, a nucleic acid sequence encoding the fusion protein of claims of the first aspect is provided.

In a third aspect, an isolated ribonucleoprotein complex is provided. The isolated ribonucleoprotein complex includes the fusion protein mutant of the first aspect and a gRNA. In a first respect, the gRNA includes a pegRNA.

In a fourth aspect, a CRISPR/Cas endonuclease system including the fusion protein mutant of the first aspect is provided. In a first respect, the CRISPR/Cas endonuclease system is encoded by a DNA expression vector. In an additional respect, the DNA expression vector is a plasmid-borne vector. In an additional respect, the DNA expression vector is selected from a bacterial expression vector and a eukaryotic expression vector.

In a fifth aspect, a method of performing gene editing in a eukaryotic cell is provided. The method includes a step of contacting a candidate editing target site locus with an active CRISPR/Cas endonuclease system having the fusion protein mutant of the first aspect

In a sixth aspect, a kit for performing gene editing in a eukaryotic cell is provided. The kit includes the fusion protein mutant of the first aspect and optionally a gRNA.

The applications of Cas9-based tools are many and varied. They include, but are not limited to: plant gene editing, yeast gene editing, mammalian gene editing, editing of cells in the organs of live animals, editing of embryos, rapid generation of knockout/knock-in animal lines, generating an animal model of disease state, correcting a disease state, inserting a reporter gene, and whole genome functional screening.

### Example 1

A bacterial prime editing selection and enrichment strategy reveals Cas9 and RTase mutations that facilitate the most potent prime editor.

Functional prime editing has recently been described in E. coli using a variety of insertions/deletions and substitutions [5]. We used expression of a prime editor and PEG-RNA that targets a kanamycin resistance gene that contains a 10-base disruption between codons 1 and 2. This prime editing strategy is designed to remove the disrupting 10-base element and restore kanamycin resistance gene expression, and thus confer resistance to the antibiotic kanamycin. The screen is setup such that a library of Cas9 Prime Editor mutants and PEG RNA are expressed from one plasmid, and the target site and non-functional kanamycin resistance gene is present on a second plasmid. Practically, bacterial cells that stably replicate the target site-containing plasmid are made competent, transformed using Cas9 prime editor and PEG RNA plasmid, and selected on Kanamycin-containing solid media.

Twenty-five saturation mutagenesis libraries (Table 1) spanning the entirety of the prime editor (Cas9 and MMLV RTase genes and the amino acid linker between the two proteins) were generated with nicking mutagenesis and the resulting library complexity was analyzed with tiled-amplicon NGS. These libraries were delivered into E. coli cells as described above to be greater than 99% confident that each possible codon change was observed at least once. The resulting colonies were pooled, plasmids from this pool were purified, and the resulting pool was sequenced with overlapping tiled amplicon NGS. Pre- and post-enrichment pools were sequenced and compared simultaneously with total read count normalization to determine enrichment for each substitution within the pool. Paired end reads were merged by fastp [6], trimmed to keep the desired mutated region by Cutadapt [7], and filtered out undesired reads with wrong length or multiple codon mutation. Codon frequency and enrichment analysis were completed by in-house python script. The results included over 32,000 amino acid substitutions that are at equal or greater frequency than the identical substitution observed in the pre-enriched pool (Table 2). Among these results are substitutions that have issued patents; however, in most cases, these patented positions are far from the most frequently observed mutations indicating the most ideal prime editing mutations are within this pool.

TABLE 1
Exemplary primers used for saturation mutagenesis
of the entire prime editor protein.

| SEQ ID | Sequence (5′-3′) |
|---|---|
| SEQ ID NO: 1 | CTAAAGAGGAGAAAGGATCTNNKGACAAAAAGTACTCTATTGGC |
| SEQ ID NO: 2 | AGAGGAGAAAGGATCTATGNNKAAAAAGTACTCTATTGGCC |
| SEQ ID NO: 3 | AGAAAGGATCTATGGACNNKAAGTACTCTATTGGCCTG |
| SEQ ID NO: 4 | AAGGATCTATGGACAAANNKTACTCTATTGGCCTGGA |
| SEQ ID NO: 5 | GGATCTATGGACAAAAAGNNKTCTATTGGCCTGGATATC |
| SEQ ID NO: 6 | CTATGGACAAAAAGTACNNKATTGGCCTGGATATCGG |
| SEQ ID NO: 7 | GACAAAAAGTACTCTNNKGGCCTGGATATCGGG |
| SEQ ID NO: 8 | GACAAAAAGTACTCTATTNNKCTGGATATCGGGACCAAC |
| SEQ ID NO: 9 | AAAGTACTCTATTGGCNNKGATATCGGGACCAACA |
| SEQ ID NO: 10 | CTCTATTGGCCTGNNKATCGGGACCAACAG |
| SEQ ID NO: 11 | TTGGCCTGGATNNKGGGACCAACAG |
| SEQ ID NO: 12 | GGCCTGGATATCNNKACCAACAGCGTC |
| SEQ ID NO: 13 | CTGGATATCGGGNNKAACAGCGTCGGG |
| SEQ ID NO: 14 | ATATCGGGACCNNKAGCGTCGGGTG |
| SEQ ID NO: 15 | TCGGGACCAACNNKGTCGGGTGGGC |
| SEQ ID NO: 16 | GGACCAACAGCNNKGGGTGGGCTGT |
| SEQ ID NO: 17 | GACCAACAGCGTCNNKTGGGCTGTTATCA |
| SEQ ID NO: 18 | ACAGCGTCGGGNNKGCTGTTATCAC |
| SEQ ID NO: 19 | GCGTCGGGTGGNNKGTTATCACCGA |
| SEQ ID NO: 20 | TCGGGTGGGCTNNKATCACCGACGA |
| SEQ ID NO: 21 | GGGTGGGCTGTTNNKACCGACGAGTATA |
| SEQ ID NO: 22 | GGGTGGGCTGTTATCNNKGACGAGTATAAAGTAC |
| SEQ ID NO: 23 | GTGGGCTGTTATCACCNNKGAGTATAAAGTACCTTC |
| SEQ ID NO: 24 | GGCTGTTATCACCGACNNKTATAAAGTACCTTCGAA |
| SEQ ID NO: 25 | CTGTTATCACCGACGAGNNKAAAGTACCTTCGAAAAA |
| SEQ ID NO: 26 | TTATCACCGACGAGTATNNKGTACCTTCGAAAAAGTTC |
| SEQ ID NO: 27 | TCACCGACGAGTATAAANNKCCTTCGAAAAAGTTCAA |
| SEQ ID NO: 28 | ACCGACGAGTATAAAGTANNKTCGAAAAAGTTCAAAGTGC |
| SEQ ID NO: 29 | CGACGAGTATAAAGTACCTNNKAAAAAGTTCAAAGTGCTGG |
| SEQ ID NO: 30 | AGTATAAAGTACCTTCGNNKAAGTTCAAAGTGCTGGG |
| SEQ ID NO: 31 | ATAAAGTACCTTCGAAANNKTTCAAAGTGCTGGGCAA |
| SEQ ID NO: 32 | GTACCTTCGAAAAAGNNKAAAGTGCTGGGCAAC |
| SEQ ID NO: 33 | TTCGAAAAAGTTCNNKGTGCTGGGCAACAC |
| SEQ ID NO: 34 | CGAAAAAGTTCAAANNKCTGGGCAACACCGAT |
| SEQ ID NO: 35 | AAAAGTTCAAAGTGNNKGGCAACACCGATCG |
| SEQ ID NO: 36 | GTTCAAAGTGCTGNNKAACACCGATCGCC |
| SEQ ID NO: 37 | AAGTGCTGGGCNNKACCGATCGCCA |
| SEQ ID NO: 38 | TGCTGGGCAACNNKGATCGCCATTC |
| SEQ ID NO: 39 | CTGGGCAACACCNNKCGCCATTCAATC |
| SEQ ID NO: 40 | CTGGGCAACACCGATNNKCATTCAATCAAAAAGA |
| SEQ ID NO: 41 | GCAACACCGATCGCNNKTCAATCAAAAAGAAC |
| SEQ ID NO: 42 | CAACACCGATCGCCATNNKATCAAAAAGAACTTGAT |
| SEQ ID NO: 43 | CACCGATCGCCATTCANNKAAAAAGAACTTGATTG |
| SEQ ID NO: 44 | CGATCGCCATTCAATCNNKAAGAACTTGATTGGTG |
| SEQ ID NO: 45 | CGCCATTCAATCAAANNKAACTTGATTGGTGCG |
| SEQ ID NO: 46 | CATTCAATCAAAAAGNNKTTGATTGGTGCGCTGT |
| SEQ ID NO: 47 | ATTCAATCAAAAAGAACNNKATTGGTGCGCTGTTGTT |
| SEQ ID NO: 48 | ATCAAAAAGAACTTGNNKGGTGCGCTGTTGTTT |
| SEQ ID NO: 49 | TCAAAAAGAACTTGATTNNKGCGCTGTTGTTTGACTC |
| SEQ ID NO: 50 | AAAGAACTTGATTGGTNNKCTGTTGTTTGACTCCG |
| SEQ ID NO: 51 | CTTGATTGGTGCGNNKTTGTTTGACTCCGG |
| SEQ ID NO: 52 | TTGGTGCGCTGNNKTTTGACTCCGG |
| SEQ ID NO: 53 | GTGCGCTGTTGNNKGACTCCGGGGA |
| SEQ ID NO: 54 | CGCTGTTGTTTNNKTCCGGGGAAACC |
| SEQ ID NO: 55 | CTGTTGTTTGACNNKGGGGAAACCGCC |
| SEQ ID NO: 56 | TTGTTTGACTCCNNKGAAACCGCCGAG |
| SEQ ID NO: 57 | TTGACTCCGGGNNKACCGCCGAGGC |
| SEQ ID NO: 58 | ACTCCGGGGAANNKGCCGAGGCGAC |
| SEQ ID NO: 59 | CCGGGGAAACCNNKGAGGCGACTCG |
| SEQ ID NO: 60 | GGGAAACCGCCNNKGCGACTCGCCT |
| SEQ ID NO: 61 | GAAACCGCCGAGNNKACTCGCCTTAAAC |
| SEQ ID NO: 62 | CCGCCGAGGCGNNKCGCCTTAAACG |
| SEQ ID NO: 63 | GCCGAGGCGACTNNKCTTAAACGTACAG |
| SEQ ID NO: 64 | AGGCGACTCGCNNKAAACGTACAGC |
| SEQ ID NO: 65 | CGACTCGCCTTNNKCGTACAGCACG |
| SEQ ID NO: 66 | ACTCGCCTTAAANNKACAGCACGTCGC |
| SEQ ID NO: 67 | GCCTTAAACGTNNKGCACGTCGCCG |
| SEQ ID NO: 68 | CCTTAAACGTACANNKCGTCGCCGGTACA |
| SEQ ID NO: 69 | TAAACGTACAGCANNKCGCCGGTACACTC |
| SEQ ID NO: 70 | GTACAGCACGTNNKCGGTACACTCGG |
| SEQ ID NO: 71 | CAGCACGTCGCNNKTACACTCGGCG |
| SEQ ID NO: 72 | CACGTCGCCGGNNKACTCGGCGTAA |
| SEQ ID NO: 73 | GTCGCCGGTACNNKCGGCGTAAGAA |
| SEQ ID NO: 74 | CGCCGGTACACTNNKCGTAAGAATCGC |
| SEQ ID NO: 75 | CCGGTACACTCGGNNKAAGAATCGCATTTG |
| SEQ ID NO: 76 | GTACACTCGGCGTNNKAATCGCATTTGCTA |
| SEQ ID NO: 77 | CACTCGGCGTAAGNNKCGCATTTGCTATTT |
| SEQ ID NO: 78 | CACTCGGCGTAAGAATNNKATTTGCTATTTGCAGG |
| SEQ ID NO: 79 | GCGTAAGAATCGCNNKTGCTATTTGCAGGA |
| SEQ ID NO: 80 | GGCGTAAGAATCGCATTNNKTATTTGCAGGAAATCTT |
| SEQ ID NO: 81 | GTAAGAATCGCATTTGCNNKTTGCAGGAAATCTTTAGC |
| SEQ ID NO: 82 | AGAATCGCATTTGCTATNNKCAGGAAATCTTTAGCAAC |
| SEQ ID NO: 83 | ATCGCATTTGCTATTTGNNKGAAATCTTTAGCAACGA |
| SEQ ID NO: 84 | GCATTTGCTATTTGCAGNNKATCTTTAGCAACGAGAT |
| SEQ ID NO: 85 | TTGCTATTTGCAGGAANNKTTTAGCAACGAGATGG |
| SEQ ID NO: 86 | ATTTGCAGGAAATCNNKAGCAACGAGATGGC |
| SEQ ID NO: 87 | ATTTGCAGGAAATCTTTNNKAACGAGATGGCAAAAGT |

TABLE 2
Exemplary amino acid changes that show potential benefit in
Prime Editing application. NGS reads were analyzed against
pre- and post-selection libraries and denoted as fold change.

| | | | | | | Post- | |
|---|---|---|---|---|---|---|---|
| | | | | | | Selection | |
| | | | | Pre- | Post- | Normalized | |
| | | | | Selection | Selection | to Pre- | Fold- |
| Target | LIB | AMP | ID | Reads | Reads | Selection | Change |
| CAS9 | 1 | 1 | D2N_GAC_AAC | [symbol] | [symbol] | [symbol] | 1.0 |
| CAS9 | 1 | 1 | D2E_GAC_GAG | 1280 | 1527 | 1345 | 1.1 |
| CAS9 | 1 | 1 | D2E_GAC_GAA | 190 | 243 | 214 | 1.1 |
| CAS9 | 1 | 1 | [symbol] | [symbol] | [symbol] | [symbol] | 1.1 |
| CAS9 | 1 | 1 | D2V_GAC_GTT | 459 | 593 | 522 | 1.1 |
| CAS9 | 1 | 1 | D2H_GAC_CAC | 95 | 126 | 111 | 1.2 |
| CAS9 | 1 | 1 | D2G_GAC_GGC | 968 | 1297 | 1142 | 1.2 |
| CAS9 | 1 | 1 | D2V_GAC_GTC | 460 | 643 | 566 | 1.2 |
| CAS9 | 1 | 1 | D2Q_GAC_CAG | 112 | 177 | 156 | 1.4 |
| CAS9 | 1 | 1 | D2D_GAC_GAT | 757 | 1759 | 1549 | 2.0 |
| CAS9 | 1 | 1 | D2A_GAC_GCT | 212 | 494 | 435 | 2.1 |
| CAS9 | 1 | 1 | D2L_GAC_CTG | 75 | 197 | 173 | 2.3 |
| CAS9 | 1 | 1 | D2A_GAC_GCG | 390 | 1255 | 1105 | 2.8 |
| CAS9 | 1 | 1 | D2R_GAC_CGG | 78 | 264 | 232 | 3.0 |
| CAS9 | 1 | 1 | [symbol] | [symbol] | [symbol] | 417 | 5.1 |
| CAS9 | 1 | 1 | D2M_GAC_ATG | 77 | 451 | 397 | 5.2 |
| CAS9 | 1 | 1 | D2S_GAC_TCT | 124 | 747 | 658 | 5.3 |
| CAS9 | 1 | 1 | [symbol] | [symbol] | [symbol] | [symbol] | [symbol] |
| CAS9 | 1 | 1 | K3K_AAA_AAG | 1344 | 1608 | 1416 | 1.1 |
| CAS9 | 1 | 1 | K3E_AAA_GAA | 869 | 1153 | 1015 | 1.2 |
| CAS9 | 1 | 1 | [symbol] | [symbol] | [symbol] | [symbol] | 1.2 |
| CAS9 | 1 | 1 | K3R_AAA_AGA | 873 | 1211 | 1066 | 1.2 |
| CAS9 | 1 | 1 | K3I_AAA_ATA | 280 | 411 | 362 | 1.3 |
| CAS9 | 1 | 1 | K3N_AAA_AAT | 424 | 628 | 553 | 1.3 |
| CAS9 | 1 | 1 | K3T_AAA_ACA | 27 | 50 | 44 | 1.6 |
| CAS9 | 1 | 1 | K3C_AAA_TGT | 116 | 217 | 191 | 1.6 |
| CAS9 | 1 | 1 | K3N_AAA_AAC | 30 | 60 | 53 | 1.8 |
| CAS9 | 1 | 1 | K3F_AAA_TTT | 174 | 361 | 318 | 1.8 |
| CAS9 | 1 | 1 | [symbol] | 101 | [symbol] | 229 | [symbol] |
| CAS9 | 1 | 1 | K3Q_AAA_CAA | 3 | 9 | 8 | 2.6 |
| CAS9 | 1 | 1 | K3V_AAA_GTT | 107 | 486 | 428 | 4.0 |
| CAS9 | 1 | 1 | [symbol] | [symbol] | [symbol] | [symbol] | 1.0 |
| CAS9 | 1 | 1 | K4S_AAG_TCG | 127 | 174 | 153 | 1.2 |
| CAS9 | 1 | 1 | K4K_AAG_AAA | 296 | 426 | 375 | 1.3 |
| CAS9 | 1 | 1 | K4N_AAG_AAC | [symbol] | [symbol] | [symbol] | [symbol] |
| CAS9 | 1 | 1 | K4G_AAG_GGG | 780 | 1633 | 1438 | 1.8 |
| CAS9 | 1 | 1 | K4R_AAG_AGA | 1 | 3 | 3 | 2.6 |
| CAS9 | 1 | 1 | Y5S_TAC_TCC | 100 | 125 | 110 | 1.1 |
| CAS9 | 1 | 1 | Y5C_TAC_TGC | 1240 | 1563 | 1376 | 1.1 |

[symbol]

indicates data missing or illegible when filed

### Example 2

Evaluating single mutants for increased prime editing activity.

The top 23 amino acid substitutions from MMLV RTase Library 1 were introduced by site-directed mutagenesis, using standard PCR conditions and primers (Table 3, SEQ ID NO: 88-149). The resulting plasmids were delivered into E. coli cells as described in Example 1. After 18 hours, the individual bacterial cells were counted and compared to the unmodified variant, denoted as PE-M63 (Table 4). Seven of the 23 substitutions resulted in a higher number of colonies, over PE-M63. These 7 substitutions are the following: S1377A, S1393A, E1406R, D1407S, E1408F, V1420F and G1423H.

The top 8 amino acid substitutions from MMLV RTase Library 1 were introduced by site-directed mutagenesis, using standard PCR conditions and primers (Table 3, SEQ ID NO: 88-149). The resulting plasmids were delivered into HEK293 cells and collected after 72 hours. The cells were lysed, the DNA extracted and amplified using primers to detect the HEK3 gene (Table 5) and treated with EcoRI to determine the percentage of prime editing. The resulting DNA was analyzed by a Fragment Analyzer and the data is summarized in Table 6. Seven of the 8 substitutions were successfully delivered into HEK293 cells. All of 7 mutants resulted in increased prime editing activity over PE-M63 and 6 of the 7 mutants resulted in a slight increase in editing over the published prime editor developed by David Liu. These substitutions are the following: S1377A, S1393A, S1397L, E1406R, D1407S, V1420F and G1423H. The only substitution from this library that is currently covered under patent claims is E1406R, and based on the editing in human cells, it is not the top performing mutant from this library. The remaining substitution, E1408F, is still in the cloning process and awaiting delivery into human cells.

TABLE 3
Primers used for saturation mutagenesis of the desired single
mutation.

| SEQ ID NO | Sequence Name | Sequence (5′-3′) |
|---|---|---|
| SEQ ID NO: | PE pACYT S1373D | GTGGGGATAGCGGTGGTAGCGATGGTGGTTCAAGCGGTAGCGAA |
| 88 | TOP | |
| SEQ ID NO: | PE pACYT S1373D | TTCGCTACCGCTTGAACCACCATCGCTACCACCGCTATCCCCAC |
| 89 | BTM | |
| SEQ ID NO: | PE pACYT S1373K | GTGGGGATAGCGGTGGTAGCAAAGGTGGTTCAAGCGGTAGCGAA |
| 90 | TOP | |
| SEQ ID NO: | PE pACYT S1373K | TTCGCTACCGCTTGAACCACCTTTGCTACCACCGCTATCCCCAC |
| 91 | BTM | |
| SEQ ID NO: | PE pACYT S1377A | GTGGTAGCAGCGGTGGTTCAGCGGGTAGCGAAACACCGGGTACAAG |
| 92 | TOP | |
| SEQ ID NO: | PE pACYT S1377A | CTTGTACCCGGTGTTTCGCTACCCGCTGAACCACCGCTGCTACCAC |
| 93 | BTM | |
| SEQ ID NO: | PE pACYT S13791 | AGCGGTGGTTCAAGCGGTATTGAAACACCGGGTACAAGCGAAAGC |
| 94 | TOP | |
| SEQ ID NO: | PE pACYT S13791 | GCTTTCGCTTGTACCCGGTGTTTCAATACCGCTTGAACCACCGCT |
| 95 | BTM | |
| SEQ ID NO: | PE pACYT P1382D | GTGGTTCAAGCGGTAGCGAAACAGATGGTACAAGCGAAAGCGCAACAC |
| 96 | TOP | |
| SEQ ID NO: | PE pACYT | GTGTTGCGCTTTCGCTTGTACCATCTGTTTCGCTACCGCTTGAACCAC |
| 97 | P1382DBTM | |
| SEQ ID NO: | PE pACYT E1386K | AGCGAAACACCGGGTACAAGCAAAAGCGCAACACCGGAAAGC |
| 98 | TOP | |
| SEQ ID NO: | PE pACYT E1386K | GCTTTCCGGTGTTGCGCTTTTGCTTGTACCCGGTGTTTCGCT |
| 99 | BTM | |
| SEQ ID NO: | PE pACYT S1393A | AGCGCAACACCGGAAAGCGCGGGTGGTAGCTCAGGTGGTAGTAGC |
| 100 | TOP | |
| SEQ ID NO: | PE pACYT S1393A | GCTACTACCACCTGAGCTACCACCCGCGCTTTCCGGTGTTGCGCT |
| 101 | BTM | |
| SEQ ID NO: | PE pACYT S1397R | CCGGAAAGCAGTGGTGGTAGCCGTGGTGGTAGTAGCACTTTAAATATT |
| 102 | TOP | GAGGATGAGC |
| SEQ ID NO: | PE pACYT S1397R | GCTCATCCTCAATATTTAAAGTGCTACTACCACCACGGCTACCACCAC |
| 103 | BTM | TGCTTTCCGG |
| SEQ ID NO: | PE pACYT G1399T | AGCAGTGGTGGTAGCTCAGGTACCAGTAGCACTTTAAATATTGAGGAT |
| 104 | TOP | GAGCATCG |
| SEQ ID NO: | PE pACYT G1399T | CGATGCTCATCCTCAATATTTAAAGTGCTACTGGTACCTGAGCTACCA |
| 105 | BTM | CCACTGCT |
| SEQ ID NO: | PE pACYT E1406R | CTCAGGTGGTAGTAGCACTTTAAATATTCGTGATGAGCATCGTTTACA |
| 106 | TOP | TGAGACATCAAA |
| SEQ ID NO: | PE pACYT E1406R | TTTGATGTCTCATGTAAACGATGCTCATCACGAATATTTAAAGTGCTA |
| 107 | BTM | CTACCACCTGAG |
| SEQ ID NO: | PE pACYT E1406S | CTCAGGTGGTAGTAGCACTTTAAATATTAGCGATGAGCATCGTTTACA |
| 108 | TOP | TGAGACATCAAA |
| SEQ ID NO: | PE pACYT E1406S | TTTGATGTCTCATGTAAACGATGCTCATCGCTAATATTTAAAGTGCTA |
| 109 | BTM | CTACCACCTGAG |
| SEQ ID NO: | PE pACYT D1407S | TCAGGTGGTAGTAGCACTTTAAATATTGAGAGCGAGCATCGTTTACAT |
| 110 | TOP | GAGACATCAAAA |
| SEQ ID NO: | PE pACYT D1407S | TTTTGATGTCTCATGTAAACGATGCTCGCTCTCAATATTTAAAGTGCT |
| 111 | BTM | ACTACCACCTGA |
| SEQ ID NO: | PE pACYT E1408F | GTGGTAGTAGCACTTTAAATATTGAGGATTITCATCGTTTACATGAGA |
| 112 | TOP | CATCAAAAGAAC |
| SEQ ID NO: | PE pACYT E1408F | GTTCTTTTGATGTCTCATGTAAACGATGAAAATCCTCAATATTTAAAG |
| 113 | BTM | TGCTACTACCAC |
| SEQ ID NO: | PE pACYT E1408L | GTGGTAGTAGCACTTTAAATATTGAGGATCTGCATCGTTTACATGAGA |
| 114 | TOP | CATCAAAAGAAC |
| SEQ ID NO: | PE pACYT E1408L | GTTCTTTTGATGTCTCATGTAAACGATGCAGATCCTCAATATTTAAAG |
| 115 | BTM | TGCTACTACCAC |
| SEQ ID NO: | PE pACYT R14101 | AGTAGCACTTTAAATATTGAGGATGAGCATATTTTACATGAGACATCA |
| 116 | TOP | AAAGAACCCGAC |
| SEQ ID NO: | PE pACYT R14101 | GTCGGGTTCTTTTGATGTCTCATGTAAAATATGCTCATCCTCAATATT |
| 117 | BTM | TAAAGTGCTACT |
| SEQ ID NO: | PE pACYT T1414Y | TTGAGGATGAGCATCGTTTACATGAGTATTCAAAAGAACCCGACGTGA |
| 118 | TOP | GCTT |
| SEQ ID NO: | PE pACYT T1414Y | AAGCTCACGTCGGGTTCTTTTGAATACTCATGTAAACGATGCTCATCC |
| 119 | BTM | TCAA |
| SEQ ID NO: | PE pACYT E1417P | GGATGAGCATCGTTTACATGAGACATCAAAACCGCCCGACGTGAGCTT |
| 120 | TOP | AGGGTC |
| SEQ ID NO: | PE pACYT E1417P | GACCCTAAGCTCACGTCGGGCGGTTTTGATGTCTCATGTAAACGATGC |
| 121 | BTM | TCATCC |
| SEQ ID NO: | PE pACYT V1420F | TCGTTTACATGAGACATCAAAAGAACCCGACTTTAGCTTAGGGTCAAC |
| 122 | TOP | GTGGCTTT |
| SEQ ID NO: | PE pACYT V1420F | AAAGCCACGTTGACCCTAAGCTAAAGTCGGGTTCTTTTGATGTCTCAT |
| 123 | BTM | GTAAACGA |
| SEQ ID NO: | PE pACYT V1420H | TCGTTTACATGAGACATCAAAAGAACCCGACCATAGCTTAGGGTCAAC |
| 124 | TOP | GTGGCTTT |
| SEQ ID NO: | PE pACYT V1420H | AAAGCCACGTTGACCCTAAGCTATGGTCGGGTTCTTTTGATGTCTCAT |
| 125 | BTM | GTAAACGA |
| SEQ ID NO: | PE pACYT S1421Y | TGAGACATCAAAAGAACCCGACGTGTATTTAGGGTCAACGTGGCTTTC |
| 126 | TOP | TGAC |
| SEQ ID NO: | PE pACYT S1421Y | GTCAGAAAGCCACGTTGACCCTAAATACACGTCGGGTTCTTTTGATGT |
| 127 | BTM | CTCA |
| SEQ ID NO: | PE pACYT G1423H | ACATCAAAAGAACCCGACGTGAGCTTACATTCAACGTGGCTTTCTGAC |
| 128 | TOP | TTCCCC |
| SEQ ID NO: | PE pACYT G1423H | GGGGAAGTCAGAAAGCCACGTTGAATGTAAGCTCACGTCGGGTTCTTT |
| 129 | BTM | TGATGT |
| SEQ ID NO: | PE pACYT G1438R | GGCGTGGGCGGAGACTCGTGGAATGGGGTTAGCTGTCCGC |
| 130 | TOP | |
| SEQ ID NO: | PE pACYT G1438R | GCGGACAGCTAACCCCATTCCACGAGTCTCCGCCCACGCC |
| 131 | BTM | |
| SEQ ID NO: | PE pACYT P1448T | GGGTTAGCTGTCCGCCAAGCAACCTTGATCATCCCGTTAAAGGCAACG |
| 132 | TOP | TC |
| SEQ ID NO: | PE pACYT P1448T | GACGTTGCCTTTAACGGGATGATCAAGGTTGCTTGGCGGACAGCTAAC |
| 133 | BTM | CC |
| SEQ ID NO: | PE pCMV E1406R | GCGGCAGCAGCACCCTAAATATAAGAGATGAGCACCGGCTACATGAGAC |
| 134 | TOP | |
| SEQ ID NO: | PE pCMV E1406R | GTCTCATGTAGCCGGTGCTCATCTCTTATATTTAGGGTGCTGCTGCCGC |
| 135 | BTM | |
| SEQ ID NO: | PE pCMV V1420F | GCTACATGAGACCTCAAAAGAGCCAGATTTCTCTCTAGGGTCCACATGG |
| 136 | TOP | CTGTC |
| SEQ ID NO: | PE pCMV V1420F | GACAGCCATGTGGACCCTAGAGAGAAATCTGGCTCTTTTGAGGTCTCAT |
| 137 | BTM | GTAGC |
| SEQ ID NO: | PE pCMV S1377A | CTGGAGGATCTAGCGGAGGATCCGCCGGCAGCGAGACACCAGGA |
| 138 | TOP | |
| SEQ ID NO: | PE pCMV S1377A | TCCTGGTGTCTCGCTGCCGGCGGATCCTCCGCTAGATCCTCCAG |
| 139 | BTM | |
| SEQ ID NO: | PE pCMV S1397L | GAGCAGTGGCGGCAGCCTGGGCGGCAGCAGCACC |
| 140 | TOP | |
| SEQ ID NO: | PE pCMV S1397L | GGTGCTGCTGCCGCCCAGGCTGCCGCCACTGCTC |
| 141 | BTM | |
| SEQ ID NO: | PE pCMV D1407S | CGGCAGCAGCACCCTAAATATAGAAAGCGAGCACCGGCTACATGAGACC |
| 142 | TOP | T |
| SEQ ID NO: | PE pCMV D1407S | AGGTCTCATGTAGCCGGTGCTCGCTTTCTATATTTAGGGTGCTGCTGCC |
| 143 | BTM | G |
| SEQ ID NO: | PE pCMV G1423H | TGAGACCTCAAAAGAGCCAGATGTTTCTCTACACTCCACATGGCTGTCT |
| 144 | TOP | GATTTTCCTCAG |
| SEQ ID NO: | PE pCMV G1423H | CTGAGGAAAATCAGACAGCCATGTGGAGTGTAGAGAAACATCTGGCTCT |
| 145 | BTM | TTTGAGGTCTCA |
| SEQ ID NO: | PE pCMV S1393A | CGAGTCAGCAACACCAGAGAGCGCCGGCGGCAGCAGCGG |
| 146 | TOP | |
| SEQ ID NO: | PE pCMV S1393A | CCGCTGCTGCCGCCGGCGCTCTCTGGTGTTGCTGACTCG |
| 147 | BTM | |
| SEQ ID NO: | PE pCMV E1408F | CGGCAGCAGCACCCTAAATATAGAAGATTTCCACCGGCTACATGAGACC |
| 148 | TOP | TCAAAAGA |
| SEQ ID NO: | PE pCMV E1408F | TCTTTTGAGGTCTCATGTAGCCGGTGGAAATCTTCTATATTTAGGGTGC |
| 149 | BTM | TGCTGCCG |

TABLE 4
Bacterial colony counts of PE-M63
and single mutant prime editors.

| Mutant | Colony Count |
|---|---|
| PE-M63 | 131 |
| S1373D | 112 |
| S1373K | 100 |
| S1377A | 250 |
| S1379I | 49 |
| P1382D | 65 |
| E1386K | 41 |
| S1393A | 207 |
| S1397R | 71 |
| G1399T | 68 |
| E1406R | 211 |
| E1406S | 115 |
| D1407S | 213 |
| E1408F | 320 |
| E1408L | 103 |
| R1410I | 92 |
| T1414Y | 21 |
| E1417P | 9 |
| V1420H | 57 |
| V1420F | 151 |
| S1421Y | 60 |
| G1423H | 222 |
| G1438R | 79 |
| P1448T | 27 |

TABLE 5
Primers used to amplify HEK3 gene from human cells.

| SEQ ID NO | Sequence Name | Sequence (5′-3′) |
|---|---|---|
| SEQ ID NO: 150 | HEK3 FWD | AGGGACGACTTTAGACCTTAGA |
| SEQ ID NO: 151 | HEK3 REV | GTCTCTGACCACTGCGATATG |

TABLE 6
Prime editing efficiency by percentage of EcoRI cleavage
by PE-M63 and single mutant prime editors after
72 hours post-delivery into HEK293 cells.

| PE Variant | % EcoRI Cleavage Triplicate | | | Average | St Dev |
|---|---|---|---|---|---|
| PE-M63 | 21 | 20.8 | 19.5 | 20.43 | 0.81 |
| David Liu PE2 | 26.2 | 25.1 | 24.6 | 25.30 | 0.82 |
| E1406R | 27.1 | 26.5 | 25.8 | 26.47 | 0.65 |
| G1423H | 28.2 | 29.3 | 30.1 | 29.20 | 0.95 |
| D1407S | 29.7 | 29.3 | 29.4 | 29.47 | 0.21 |
| S1397L | 29.6 | 29.6 | 29.8 | 29.67 | 0.12 |
| S1377A | 29.2 | 31.1 | 28.9 | 29.73 | 1.19 |
| S1393A | 30.5 | 29.7 | 29.3 | 29.83 | 0.61 |
| V1420F | 32.5 | 29.7 | 28.4 | 30.20 | 2.10 |
| Cells Alone | 0 | 0 | 0 | 0.00 | 0.00 |

### Example 3

Additional Cas9 and RTase mutations were identified with a revised bacterial prime editing selection and enrichment strategy using a modified strategy as described in Examples 1 and 2.

Functional prime editing has recently been described in E. coli using a variety of insertions/deletions and substitutions [5]. We used expression of a prime editor and PEG-RNA that targets a kanamycin resistance gene that contains a 10-base disruption between codons 1 and 2. This prime editing strategy is designed to remove the disrupting 10-base element and restore kanamycin resistance gene expression, and thus confer resistance to the antibiotic kanamycin. The screen was modified such that a library of Cas9 Prime Editor mutants and PEG RNA are expressed from one plasmid, and the target site and non-functional kanamycin resistance gene is present on the chromosome. Practically, bacterial cells that repair and stably replicate the kanamycin resistance gene are made competent, transformed using Cas9 prime editor and PEG RNA plasmid, and selected on Kanamycin-containing solid media.

Twenty-five saturation mutagenesis libraries (Table 1) spanning the entirety of the prime editor (Cas9 and MMLV RTase genes and the amino acid linker between the two proteins) were generated with nicking mutagenesis and the resulting library complexity was analyzed with tiled-amplicon NGS. These libraries were delivered into E. coli cells as described above to be greater than 99% confident that each possible codon change was observed at least once. The resulting colonies were pooled, plasmids from this pool were purified, and supplied back into the same E. coli cells for subsequent rounds and pooling, until E. coli cells reached saturation (up to five rounds). The resulting pools from each round were sequenced with overlapping tiled amplicon NGS. Pre- and post-enrichment pools were sequenced and compared simultaneously with total read count normalization to determine enrichment for each substitution within the pool. Paired end reads were merged by fastp [6], trimmed to keep the desired mutated region by Cutadapt [7], and filtered out undesired reads with wrong length or multiple codon mutation. Codon frequency and enrichment analysis were completed by in-house python script. For M63 RTase libraries 1, 2, 4 and 5, the results included over 1,200 amino acid substitutions that are at equal or greater frequency than the identical substitution observed in the pre-enriched pool (Table 7). Among these results are substitutions that have issued patents; however, in most cases, these patented positions are far from the most frequently observed mutations indicating the most ideal prime editing mutations are within this pool. The top 10 substitutions from each library from Table 2 will be cloned and tested in human cells for prime editing efficiency (denoted in Table 3).

TABLE 7
Amino acid changes that show potential benefit in Prime Editing
application. NGS reads were analyzed against pre- and post-
selection libraries and denoted as fold change.

| | | | Fold- | Fold- | Fold- | Fold- | Fold- |
|---|---|---|---|---|---|---|---|
| M63 | | | Change | Change | Change | Change | Change |
| Library | Position | Mutation | Round 1 | Round 2 | Round 3 | Round 4 | Round 5 |
| 1 | 1412 | H1412A | 4.6 | 36.1 | 44.0 | 194.0 | 434.7 |
| 1 | 1421 | S1421P | 3.5 | 57.1 | 202.3 | 392.9 | 337.4 |
| 1 | 1412 | H1412I | 2.6 | 42.3 | 76.9 | 158.2 | 144.0 |
| 1 | 1386 | E1386L | 1.3 | 18.2 | 49.2 | 104.6 | 108.3 |
| 1 | 1440 | M1440G | 1.3 | 6.9 | 24.3 | 39.4 | 74.1 |
| 1 | 1423 | G1423Q | 3.8 | 21.5 | 53.9 | 75.7 | 64.3 |
| 1 | 1419 | D1419Q | 0.8 | 13.3 | 55.9 | 69.5 | 53.6 |
| 1 | 1406 | E1406L | 0.8 | 8.7 | 13.7 | 49.7 | 51.4 |
| 1 | 1400 | S1400T | 2.9 | 25.3 | 22.0 | 56.6 | 35.8 |
| 1 | 1381 | T1381L | 2.0 | 14.1 | 21.1 | 54.2 | 35.0 |
| 2 | 1521 | K1521V | 1.0 | 4.7 | 38.9 | 244.6 | N/A |
| 2 | 1480 | R1480P | 0.3 | 1.4 | 17.9 | 123.1 | N/A |
| 2 | 1507 | T1507W | 1.2 | 2.9 | 12.4 | 53.1 | N/A |
| 2 | 1500 | R1500Y | 2.2 | 10.4 | 18.8 | 16.8 | N/A |
| 2 | 1504 | K1504P | 1.6 | 11.3 | 16.0 | 13.1 | N/A |
| 2 | 1458 | T1458R | 3.6 | 16.0 | 18.1 | 12.8 | N/A |
| 2 | 1524 | E1524P | 2.5 | 4.5 | 8.1 | 12.5 | N/A |
| 2 | 1491 | C1491H | 1.6 | 10.8 | 17.3 | 12.0 | N/A |
| 2 | 1470 | E1470S | 0.5 | 8.3 | 15.6 | 11.7 | N/A |
| 2 | 1505 | P1505T | 1.5 | 9.0 | 12.9 | 10.9 | N/A |
| 2 | 1458 | T1458D | 2.6 | 6.4 | 11.7 | 10.7 | N/A |
| 4 | 1646 | Q1646R | 3.1 | 12.8 | 113.4 | 207.5 | 92.7 |
| 4 | 1660 | A1660S | 0.4 | 1.2 | 0.3 | 84.0 | 87.8 |
| 4 | 1680 | W1680Q | 1.1 | 6.4 | 53.5 | 59.3 | 82.3 |
| 4 | 1622 | Q1622K | 0.9 | 1.5 | 18.8 | 50.1 | 49.0 |
| 4 | 1638 | Q1638S | 1.2 | 3.3 | 19.9 | 20.0 | 37.2 |
| 4 | 1632 | T1632A | 0.7 | 2.0 | 8.2 | 26.2 | 30.7 |
| 4 | 1680 | W1680N | 0.6 | 1.1 | 10.9 | 22.8 | 30.4 |
| 4 | 1670 | L1670L | 0.7 | 0.9 | 13.5 | 35.8 | 29.3 |
| 4 | 1629 | L1629L | 1.3 | 2.5 | 29.8 | 38.0 | 27.9 |
| 4 | 1627 | L1627I | 1.1 | 1.4 | 19.9 | 25.0 | 23.2 |
| 5 | 1741 | Q1741P | 1.6 | 44.9 | 35.2 | 51.7 | N/A |
| 5 | 1764 | T1764N | 2.3 | 13.7 | 47.2 | 51.0 | N/A |
| 5 | 1734 | L1734S | 1.5 | 26.5 | 37.9 | 37.9 | N/A |
| 5 | 1734 | L1734E | 2.7 | 13.1 | 10.3 | 25.4 | N/A |
| 5 | 1743 | K1743C | 2.5 | 6.2 | 17.8 | 25.2 | N/A |
| 5 | 1754 | T1754S | 2.0 | 5.7 | 25.3 | 25.1 | N/A |
| 5 | 1760 | L1760I | 1.2 | 6.5 | 19.3 | 23.1 | N/A |
| 5 | 1750 | Q1750E | 1.2 | 1.6 | 16.4 | 21.2 | N/A |
| 5 | 1766 | P1766R | 1.4 | 20.1 | 32.1 | 17.9 | N/A |
| 5 | 1703 | E1703V | 0.7 | 10.2 | 18.6 | 17.5 | N/A |

### Example 4

Additional Cas9 and RTase mutations were identified with a revised bacterial prime editing selection and enrichment strategy using the modified strategy of Example 3. For M63 RTase libraries 3, 6, 7, 8 and 9, the results included 389 amino acid substitutions that are at equal or greater frequency than the identical substitution observed in the pre-enriched pool (Table 8). Among these results are substitutions that have issued patents; however, in most cases, these patented positions are far from the most frequently observed mutations indicating the most ideal prime editing mutations are within this pool. The top 10 substitutions from each library from Table 8 will be cloned and tested in human cells for prime editing efficiency (denoted in Table 8).

TABLE 8
Mutant candidates for increased Prime editing
efficiency from M63 Lib 3 6 7 8 9

| M63 | | | Fold- | Fold- | Fold- | Fold- |
|---|---|---|---|---|---|---|
| Li- | Posi- | Muta- | Change | Change | Change | Change |
| brary | tion | tion | Round 1 | Round 2 | Round 3 | Round 4 |
| 3 | 1560 | R1560A | 4.9 | 2031.4 | 3478.2 | N/A |
| 3 | 1560 | R1560P | 0.6 | 11.5 | 19.3 | N/A |
| 3 | 1536 | L1536V | 3.0 | 3.3 | 2.7 | N/A |
| 3 | 1542 | P1542K | 2.0 | 0.7 | 1.1 | N/A |
| 3 | 1556 | F1556A | 0.5 | 4.7 | 1.0 | N/A |
| 3 | 1560 | R1560G | 1.6 | 0.7 | 0.9 | N/A |
| 3 | 1580 | I1580L | 2.9 | 1.6 | 0.8 | N/A |
| 3 | 1573 | W1573S | 0.6 | 0.7 | 0.8 | N/A |
| 3 | 1560 | R1560S | 0.8 | 0.6 | 0.8 | N/A |
| 3 | 1580 | I1580T | 1.6 | 0.7 | 0.8 | N/A |
| 6 | 1829 | M1829A | 0.7 | 0.2 | 7.0 | N/A |
| 6 | 1786 | L1786P | 0.5 | 0.4 | 6.6 | N/A |
| 6 | 1830 | G1830L | 4.0 | 7.1 | 5.8 | N/A |
| 6 | 1841 | V1841P | 1.4 | 3.8 | 4.2 | N/A |
| 6 | 1836 | L1836H | 10.9 | 5.7 | 3.5 | N/A |
| 6 | 1806 | G1806T | 1.3 | 2.4 | 2.9 | N/A |
| 6 | 1771 | V1771T | 2.6 | 2.9 | 2.8 | N/A |
| 6 | 1783 | T1783H | 1.0 | 2.7 | 2.7 | N/A |
| 6 | 1828 | T1828G | 1.2 | 2.9 | 2.5 | N/A |
| 6 | 1825 | G1825Y | 0.2 | 1.4 | 2.3 | N/A |
| 7 | 1879 | L1879A | 8.7 | 6274.0 | 8364.0 | N/A |
| 7 | 1879 | L1879D | 0.6 | 8.1 | 10.7 | N/A |
| 7 | 1875 | P1875N | 7.1 | 12.6 | 8.8 | N/A |
| 7 | 1925 | G1925P | 6.7 | 5.3 | 3.4 | N/A |
| 7 | 1891 | G1891T | 14.3 | 10.6 | 3.2 | N/A |
| 7 | 1883 | T1883R | 3.7 | 3.4 | 2.8 | N/A |
| 7 | 1891 | G1891A | 0.3 | 3.4 | 2.8 | N/A |
| 7 | 1879 | L1879Y | 1.4 | 2.7 | 2.5 | N/A |
| 7 | 1879 | L1879S | 0.0 | 2.6 | 2.4 | N/A |
| 7 | 1891 | G1891N | 11.9 | 5.6 | 2.0 | N/A |
| 7 | 1879 | L1879F | 1.4 | 2.4 | 1.8 | N/A |
| 8 | 1971 | L1971R | 1.2 | 1.4 | 1.6 | N/A |
| 8 | 1952 | A1952P | 1.2 | 1.5 | 1.6 | N/A |
| 8 | 1980 | N1980H | 1.2 | 1.4 | 1.6 | N/A |
| 8 | 1988 | A1988G | 1.7 | 0.4 | 1.4 | N/A |
| 8 | 2002 | R2002S | 1.1 | 1.2 | 1.3 | N/A |
| 8 | 1963 | Q1963P | 1.1 | 1.2 | 1.2 | N/A |
| 8 | 1992 | A1992P | 0.8 | 0.8 | 1.1 | N/A |
| 8 | 1963 | Q1963L | 0.8 | 1.0 | 1.1 | N/A |
| 8 | 1993 | H1993P | 1.2 | 1.4 | 1.1 | N/A |
| 8 | 1972 | K1972M | 0.6 | 0.8 | 1.1 | N/A |
| 9 | 2014 | N2014C | 0.5 | 34.7 | 140.8 | 120.5 |
| 9 | 2062 | I2062P | 1.0 | 5.6 | 4.8 | 99.9 |
| 9 | 2047 | A2047V | 0.9 | 3.6 | 4.1 | 74.4 |
| 9 | 2037 | P2037T | 3.0 | 23.4 | 91.6 | 50.3 |
| 9 | 2029 | K2029Q | 5.1 | 45.5 | 43.4 | 43.2 |
| 9 | 2043 | H2043P | 3.2 | 42.7 | 38.1 | 34.1 |
| 9 | 2025 | L2025W | 0.9 | 7.2 | 19.5 | 32.1 |
| 9 | 2044 | S2044R | 1.1 | 25.7 | 33.7 | 30.1 |
| 9 | 2030 | R2030M | 0.3 | 29.7 | 28.1 | 28.5 |
| 9 | 2042 | G2042R | 1.6 | 6.1 | 30.0 | 28.0 |

### Example 5

Additional Cas9 and RTase mutations were identified with a revised bacterial prime editing selection and enrichment strategy using the modified strategy of Example 3. For Cas9 libraries 1-8, the results included 262 amino acid substitutions that are at equal or greater frequency than the identical substitution observed in the pre-enriched pool (Table 9). Among these results are substitutions that have issued patents; however, in most cases, these patented positions are far from the most frequently observed mutations indicating the most ideal prime editing mutations are within this pool. The top 5 substitutions from each library from Table 2 will be cloned and tested in human cells for prime editing efficiency (denoted in Table 9).

TABLE 9
Mutant candidates for increased Prime
editing efficiency for Cas9 Lib1-8

| | | | Fold- | Fold- | Fold- |
|---|---|---|---|---|---|
| Cas9 | | | Change | Change | Change |
| Library | Position | Mutation | Rd 1 | Rd 2 | Rd 3 |
| 1 | 53 | F53C | 0.63 | 48.26 | 16.28 |
| 1 | 31 | K31Q | 2.01 | 1.06 | 2.41 |
| 1 | 79 | I79T | 0.71 | 1.59 | 1.57 |
| 1 | 50 | A50P | 0.91 | 1.54 | 1.53 |
| 1 | 97 | F97Y | 0.70 | 1.57 | 1.49 |
| 2 | 175 | N175A | 13.16 | 20.98 | 3.11 |
| 2 | 197 | E197R | 8.72 | 3.89 | 2.52 |
| 2 | 185 | F185STOP | 19.05 | 9.56 | 2.27 |
| 2 | 104 | S104STOP | 7.11 | 7.94 | 2.09 |
| 2 | 195 | L195P | 5.57 | 7.01 | 1.79 |
| 3 | 271 | Y271G | 5.43 | 2347.92 | 2361.72 |
| 3 | 275 | L275A | 4.32 | 47.49 | 39.25 |
| 3 | 239 | G239L | 16.68 | 20.01 | 14.76 |
| 3 | 264 | L264T | 22.93 | 7.40 | 5.82 |
| 3 | 271 | Y271R | 0.04 | 6.00 | 4.37 |
| 4 | 385 | G385S | 19.00 | 7.08 | 3.11 |
| 4 | 346 | K346N | 1.64 | 2.35 | 2.38 |
| 4 | 325 | Y325F | 1.32 | 1.25 | 1.30 |
| 4 | 308 | V308D | 1.08 | 1.10 | 1.28 |
| 4 | 326 | D326E | 1.26 | 1.38 | 1.26 |
| 5 | 446 | F446L | 11.32 | 3.81 | 1.44 |
| 5 | 441 | E441A | 1.26 | 0.99 | 1.43 |
| 5 | 442 | K442STOP | 1.15 | 1.23 | 1.39 |
| 5 | 437 | R437Q | 1.00 | 1.30 | 1.35 |
| 5 | 471 | E471D | 1.21 | 1.08 | 1.31 |
| 6 | 581 | S581STOP | 10.76 | 2355.42 | 2045.97 |
| 6 | 518 | F518\| | 1.03 | 1.03 | 1.61 |
| 6 | 502 | L502M | 1.04 | 1.16 | 1.37 |
| 6 | 517 | Y517N | 0.98 | 1.00 | 1.28 |
| 6 | 516 | E516Q | 48.69 | 7.34 | 1.18 |
| 7 | 674 | Q674H | 1.54 | 1346.54 | 1471.33 |
| 7 | 654 | R654D | 17.73 | 6.70 | 7.14 |
| 7 | 603 | D603V | 28.78 | 15.15 | 4.70 |
| 7 | 674 | Q674L | 0.02 | 3.55 | 3.23 |
| 7 | 616 | L616R | 9.90 | 7.16 | 2.85 |
| 8 | 741 | V741G | 0.80 | 317.22 | 349.07 |
| 8 | 776 | N776A | 8.98 | 5.69 | 1.69 |
| 8 | 775 | K775S | 1.65 | 9.06 | 1.44 |
| 8 | 771 | N776Q | 15.07 | 5.16 | 0.88 |
| 8 | 775 | H799Y | 12.61 | 5.33 | 0.71 |

### Example 6

Additional Cas9 and RTase mutations were identified with a revised bacterial prime editing selection and enrichment strategy using the modified strategy of Example 3. For Cas9 libraries 9-15, the results included over 2,900 amino acid substitutions that are at equal or greater frequency than the identical substitution observed in the pre-enriched pool (Table 10). Among these results are substitutions that have issued patents; however, in most cases, these patented positions are far from the most frequently observed mutations indicating the most ideal prime editing mutations are within this pool. The top substitutions from each library from Table 10 will be cloned and tested in human cells for prime editing efficiency.

TABLE 10
Mutant candidates for increased Prime
editing efficiency for Cas9 Lib9-15.

| | | | Fold- | Fold- | Fold- |
|---|---|---|---|---|---|
| Cas9 | | | Change | Change | Change |
| Library | Position | Mutation | Rd 1 | Rd 2 | Rd 3 |
| 9 | 888 | N888L | 30.0 | 24.0 | 14.1 |
| 9 | 874 | E874T | 19.7 | 3.5 | 3.6 |
| 9 | 863 | N863A | 30.1 | 10.2 | 2.6 |
| 9 | 881 | N881H | 35.4 | 4.3 | 1.7 |
| 10 | 939 | M939V | 7.9 | 2244.1 | 2586.4 |
| 10 | 905 | R905N | 80.5 | 0.0 | 3.2 |
| 10 | 991 | A991STOP | 13.4 | 2.4 | 1.4 |
| 10 | 915 | G915P | 41.0 | 3.4 | 1.1 |
| 11 | 1074 | W1074S | 4.0 | 1681.8 | 886.9 |
| 11 | 1046 | F1046S | 7.9 | 3.3 | 3.4 |
| 11 | 1024 | K1024A | 15.4 | 0.0 | 2.1 |
| 12 | 1105 | F1105G | 5.8 | 142.3 | 96.4 |
| 12 | 1125 | D1125E | 0.2 | 0.2 | 4.1 |
| 13 | 1278 | K1278I | 127.6 | 10233.8 | 11443.4 |
| 13 | 1273 | I1273Y | 11.1 | 478.7 | 199.1 |
| 13 | 1261 | Q1261S | 126.4 | 11.1 | 4.6 |
| 13 | 1217 | A1217F | 95.5 | 7.7 | 1.5 |
| 13 | 1244 | K1244L | 51.4 | 9.2 | 1.4 |
| 13 | 1219 | E1219M | 40.9 | 3.9 | 1.3 |
| 13 | 1260 | E1260S | 45.6 | 6.0 | 0.7 |
| 14 | 1329 | T1329A | 3.3 | 505.1 | 619.4 |
| 14 | 1396 | S1396C | 4.4 | 204.8 | 219.0 |
| 14 | 1380 | E1380T | 145.5 | 10.4 | 2.3 |
| 14 | 1354 | G1354N | 29.4 | 10.2 | 1.1 |
| 15 | 487 | S487P | 22.4 | 1984.8 | 1142.2 |
| 15 | 608 | D608L | 28.4 | 1552.9 | 982.9 |
| 15 | 1043 | M1043A | 12.4 | 44.6 | 33.6 |

### Example 7

Additional Cas9 and RTase mutations were identified with a revised bacterial prime editing selection and enrichment strategy using the modified strategy of Example 3. For Cas9 and M63 libraries, additional Mutant ID's having increased Prime editing efficiency were identified.

### Example 8

### Amino Acid and Nucleic Acid Sequences

The following amino acid and nucleic acid sequences support this disclosure.

SpyCas9 (H840A) protein AA sequence
SEQ ID NO: 152
MDKKYSIGLDIGTNSVGWAVITDEYKVPSKKFKVLGNTDRHSIKKNLIGALLEDSGETAEATRLKRTARRRYTR
RKNRICYLQEIFSNEMAKVDDSFFHRLEESFLVEEDKKHERHPIFGNIVDEVAYHEKYPTIYHLRKKLVDSTDK
ADLRLIYLALAHMIKFRGHFLIEGDLNPDNSDVDKLFIQLVQTYNQLFEENPINASGVDAKAILSARLSKSRRL
ENLIAQLPGEKKNGLFGNLIALSLGLTPNFKSNFDLAEDAKLQLSKDTYDDDLDNLLAQIGDQYADLFLAAKNL
SDAILLSDILRVNTEITKAPLSASMIKRYDEHHQDLTLLKALVRQQLPEKYKEIFFDQSKNGYAGYIDGGASQE
EFYKFIKPILEKMDGTEELLVKLNREDLLRKQRTFDNGSIPHQIHLGELHAILRRQEDFYPFLKDNREKIEKIL
TFRIPYYVGPLARGNSRFAWMTRKSEETITPWNFEEVVDKGASAQSFIERMTNFDKNLPNEKVLPKHSLLYEYE
TVYNELTKVKYVTEGMRKPAFLSGEQKKAIVDLLFKTNRKVTVKQLKEDYFKKIECFDSVEISGVEDRENASLG
TYHDLLKIIKDKDFLDNEENEDILEDIVLTLTLFEDREMIEERLKTYAHLFDDKVMKQLKRRRYTGWGRLSRKL
INGIRDKQSGKTILDFLKSDGFANRNFMQLIHDDSLTFKEDIQKAQVSGQGDSLHEHIANLAGSPAIKKGILQT
VKVVDELVKVMGRHKPENIVIEMARENQTTQKGQKNSRERMKRIEEGIKELGSQILKEHPVENTQLQNEKLYLY
YLQNGRDMYVDQELDINRLSDYDVDAIVPQSFLKDDSIDNKVLTRSDKNRGKSDNVPSEEVVKKMKNYWRQLLN
AKLITQRKFDNLTKAERGGLSELDKAGFIKRQLVETRQITKHVAQILDSRMNTKYDENDKLIREVKVITLKSKL
VSDFRKDFQFYKVREINNYHHAHDAYLNAVVGTALIKKYPKLESEFVYGDYKVYDVRKMIAKSEQEIGKATAKY
FFYSNIMNFFKTEITLANGEIRKRPLIETNGETGEIVWDKGRDFATVRKVLSMPQVNIVKKTEVQTGGFSKESI
LPKRNSDKLIARKKDWDPKKYGGFDSPTVAYSVLVVAKVEKGKSKKLKSVKELLGITIMERSSFEKNPIDFLEA
KGYKEVKKDLIIKLPKYSLFELENGRKRMLASAGELQKGNELALPSKYVNFLYLASHYEKLKGSPEDNEQKQLF
VEQHKHYLDEIIEQISEFSKRVILADANLDKVLSAYNKHRDKPIREQAENIIHLFTLTNLGAPAAFKYFDTTID
RKRYTSTKEVLDATLIHQSITGLYETRIDLSQLGGD
WT MMLV protein AA sequence
SEQ ID NO: 153
TLNIEDEYRLHETSKEPDVSLGSTWLSDFPQAWAETGGMGLAVRQAPLIIPLKATSTPVSIKQYPMSQEARLGI
KPHIQRLLDQGILVPCQSPWNTPLLPVKKPGTNDYRPVQDLREVNKRVEDIHPTVPNPYNLLSGLPPSHQWYTV
LDLKDAFFCLRLHPTSQPLFAFEWRDPEMGISGQLTWTRLPQGFKNSPTLFDEALHRDLADFRIQHPDLILLQY
VDDLLLAATSELDCQQGTRALLQTLGNLGYRASAKKAQICQKQVKYLGYLLKEGQRWLTEARKETVMGQPTPKT
PRQLREFLGTAGFCRLWIPGFAEMAAPLYPLIKTGTLFNWGPDQQKAYQEIKQALLTAPALGLPDLTKPFELFV
DEKQGYAKGVLTQKLGPWRRPVAYLSKKLDPVAAGWPPCLRMVAAIAVLTKDAGKLTMGQPLVILAPHAVEALV
KQPPDRWLSNARMTHYQALLLDTDRVQFGPVVALNPATLLPLPEEGLQHNCLDILAEAHGTRPDLTDQPLPDAD
HTWYTDGSSLLQEGQRKAGAAVTTETEVIWAKALPAGTSAQRAELIALTQALKMAEGKKLNVYTDSRYAFATAH
IHGEIYRRRGLLTSEGKEIKNKDEILALLKALFLPKRLSIIHCPGHQKGHSAEARGNRMADQAARKAAITETPD
TSTLLIENSSP
Mutant MMLV-II protein AA sequence
SEQ ID NO: 154
TLNIEDEYRLHETSKEPDVSLGSTWLSDFPQAWAETGGMGLAVRQAPLIIPLKATSTPVSIKQYPMSQEARLGI
KPHIQRLLDQGILVPCQSPWNTPLLPVKKPGTNDYRPVQDLREVNKRVEDIHPTVPNPYNLLSGLPPSHQWYTV
LDLKDAFFCLRLHPTSQPLFAFEWRDPEMGISGQLTWTRLPQGFKNSPTLFDEALHRDLADFRIQHPDLILLQY
VDDLLLAATSELDCQQGTRALLQTLGNLGYRASAKKAQICQKQVKYLGYLLKEGQRWLTEARKETVMGQPTPKT
PRQLREFLGTAGFCRLWIPGFAEMAAPLYPLTKTGTLFNWGPDQQKAYQEIKQALLTAPALGLPDLTKPFELFV
DEKQGYAKGVLTQKLGPWRRPVAYLSKKLDPVAAGWPPCLRMVAAIAVLTKDAGKLTMGQPLVILAPHAVEALV
KQPPDRWLSNARMTHYQALLLDTDRVQFGPVVALNPATLLPLPEEGLQHNCLDILAEAHGTRPDLTDQPLPDAD
HTWYTGGSSLLQEGQRKAGAAVTTETEVIWAKALPAGTSAQRAQLIALTQALKMAEGKKLNVYTNSRYAFATAH
IHGEIYRRRGLLTSEGKEIKNKDEILALLKALFLPKRLSIIHCPGHQKGHSAEARGNRMADQAARKAAITETPD
TSTLLIENSSP
Mutant PE2 MMLV RT protein AA sequence
SEQ ID NO: 155
TLNIEDEYRLHETSKEPDVSLGSTWLSDFPQAWAETGGMGLAVRQAPLIIPLKATSTPVSIKQYPMSQEARLGI
KPHIQRLLDQGILVPCQSPWNTPLLPVKKPGTNDYRPVQDLREVNKRVEDIHPTVPNPYNLLSGLPPSHQWYTV
LDLKDAFFCLRLHPTSQPLFAFEWRDPEMGISGQLTWTRLPQGFKNSPTLFNEALHRDLADFRIQHPDLILLQY
VDDLLLAATSELDCQQGTRALLQTLGNLGYRASAKKAQICQKQVKYLGYLLKEGQRWLTEARKETVMGQPTPKT
PRQLREFLGKAGFCRLFIPGFAEMAAPLYPLTKPGTLFNWGPDQQKAYQEIKQALLTAPALGLPDLTKPFELFV
DEKQGYAKGVLTQKLGPWRRPVAYLSKKLDPVAAGWPPCLRMVAAIAVLTKDAGKLTMGQPLVILAPHAVEALV
KQPPDRWLSNARMTHYQALLLDTDRVQFGPVVALNPATLLPLPEEGLQHNCLDILAEAHGTRPDLTDQPLPDAD
HTWYTDGSSLLQEGQRKAGAAVTTETEVIWAKALPAGTSAQRAELIALTQALKMAEGKKLNVYTDSRYAFATAH
IHGEIYRRRGWLTSEGKEIKNKDEILALLKALFLPKRLSIIHCPGHQKGHSAEARGNRMADQAARKAAITETPD
TSTLLIENSSP
Mutant M63 protein AA sequence
SEQ ID NO: 156
TLNIEDEYRLHETSKEPDVSLGSTWLSDFPQAWAETGGMGLAVRQAPLIIPLKATSTPVSIKQYPMSREARLGI
KPHIRRLYDQGILVPCQSPWNTPLRPVKKPGTNDYRPVQDLREVNKRVEDIHPTVPNPYNLLSGLPPSHQWYTV
LDLKDAFFCLRLHPTSQPLFAFEWRDPEMGISGQLTWTRLPQGFKNSPTLFDEALHRDLADFRIQHPDLILLQY
VDDLLLAATSELDCQQGTRALLQTLGNLGYRASAKKAQICQKQVKYLGYLLKEGQRWITDARKETVMGQPTPKT
PRELREFLGKAGFCRLWIPGFAEMAAPLYPLIKTGTLFNWGPDQQKAYQEIKQALLTAPALGLPDLTKPFELFV
DEKQGYAKGVLTQKLGPWRRPVAYLSKKLDPVAAGWPPCLRMVAAIAVLTKDAGKLTMGQPLNILAPHAVEALV
KQPPDRWLSNARMTHYQALLLDTDRVQFGPVVALNPATLLPLPEEGLQHNCLDILAEAHGTRPDLTDQPLPDAD
HTWYTGGSSLLQEGQRKAGAAVTTETEVIWAKALPAGTSAQRAQLIALTQALKMAEGKKLNVYTNSRYAFATAH
WHGEIYRRRGLLTSEGKEIKNKDEILALLKALFLPKRLSIIHCPGHQKGHSAEARGNRMADQAARKAAITETPD
TSTLLIENSSP

### REFERENCES

1. Jinek, M., et al., A programmable dual-RNA-guided DNA endonuclease in adaptive bacterial immunity. Science, 2012. 337 (6096): p. 816-21.
2. Komor, A. C., et al., Programmable editing of a target base in genomic DNA without double-stranded DNA cleavage. Nature, 2016. 533 (7603): p. 420-4.
3. Gaudelli, N. M., et al., Programmable base editing of A*T to G*C in genomic DNA without DNA cleavage. Nature, 2017. 551 (7681): p. 464-471.
4. Anzalone, A. V., et al., Search-and-replace genome editing without double-strand breaks or donor DNA. Nature, 2019. 576 (7785): p. 149-157.
5. Tong, Y., et al., A versatile genetic engineering toolkit for E. coli based on CRISPR-prime editing. Nat Commun, 2021. 12 (1): p. 5206.
6. Chen, S., et al., fastp: an ultra-fast all-in-one FASTQ preprocessor. Bioinformatics, 2018. 34 (17): p. 884-890.
7. Martin M. Cutadapt removes adapter sequences from high-throughput sequencing reads. EMBnet. journal. 2011. 17 (1): p. 10-2.

All references, including publications, patent applications, and patents cited herein are hereby incorporated by reference to the same extent as if each reference were individually and specifically indicated to be incorporated by reference and were set forth in its entirety herein.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A fusion protein mutant comprising a Prime Editing enzyme having a first amino acid sequence and a second amino acid sequence, wherein the first amino acid sequence comprises a SpCas9 H840A nickase mutant protein of SEQ ID NO: 152 and the second amino acid sequence comprises a Moloney Murine Leukemia Virus reverse transcriptase protein mutant (MMLV RTase mutant), wherein the fusion protein mutant displays at least the equivalent or greater activity of a reference Prime Editing enzyme in genome editing.

2. The fusion protein mutant of claim 1, comprising a member of the group selected from one of Tables 2, 6, 7, 8, 9, 10, and 11.

3. A nucleic acid sequence encoding the fusion protein of claim 1.

4. An isolated ribonucleoprotein complex, wherein the isolated ribonucleoprotein complex comprises the fusion protein of claim 1 and a gRNA.

5. The isolated ribonucleoprotein complex of claim 5, wherein the gRNA comprises a pegRNA.

6. A CRISPR/Cas endonuclease system comprising the fusion protein of claim 1.

7. The CRISPR/Cas endonuclease system of claim 7, wherein the CRISPR/Cas endonuclease system is encoded by a DNA expression vector.

8. The CRISPR/Cas endonuclease system of claim 8, the DNA expression vector is a plasmid-borne vector.

9. The CRISPR/Cas endonuclease system of claim 9, wherein the DNA expression vector is selected from a bacterial expression vector and a eukaryotic expression vector.

10. A method of performing gene editing in a eukaryotic cell, comprising a step of contacting a candidate editing target site locus with an active CRISPR/Cas endonuclease system having the fusion protein of claim 1.

11. A kit for performing gene editing in a eukaryotic cell, wherein the kit comprises the fusion protein of claim 1 and optionally a gRNA.
